(19) United States
(12) Patent Application Publication
(10) Pub. No.: US 2025/0327799 A1
(43) Pub. Date: Oct. 23, 2025

(54) ASSESSING AND TREATING CAVEOLINOPATHY DISEASES

(71) Applicant: Mayo Foundation for Medical Education and Research, Rochester, MN (US)

(72) Inventors: Divyanshu Dubey, Rochester, MN (US); Sean J. Pittock, Rochester, MN (US); Andrew M. Knight, Stewartville, MN (US); Margherita Milone, Rochester, MN (US); M. Bakri Hammami, Bronx, NY (US)

(21) Appl. No.: 18/864,114

(22) PCT Filed: May 15, 2023
(86) PCT No.: PCT/US2023/022247

§ 371 Date: Nov. 8, 2024

Related U.S. Application Data
(60) Provisional application No. 63/342,397, filed on May 16, 2022.

(51) Int. Cl. G01N 33/564

(52) U.S. Cl. CPC G01N 33/564; G01N 2800/10; G01N 2800/52

(57) ABSTRACT

This document relates to methods and materials involved in assessing and/or treating mammals having a caveolinopathy disease (e.g., immune-mediated rippling muscle disease (iRMD)). For example, methods and materials for detecting the presence of an autoantibody specific for a caveolae-associated protein 4 (cavin-4) polypeptide to identify a mammal as having iRMD as well as methods for treating a mammal identified having iRMD are provided. In some cases, cavin-4 polypeptides (and/or fragments thereof and/or variants thereof) can be used to detect the presence or absence of anti-cavin-4 polypeptide autoantibodies indicative of iRMD.

### CROSS-REFERENCE TO RELATED APPLICATIONS

This application claims the benefit of U.S. Patent Application Ser. No. 63/342,397, filed on May 16, 2022. The disclosure of the prior application is considered part of, and is incorporated by reference in, the disclosure of this application.

### SEQUENCE LISTING

This application contains a Sequence Listing that has been submitted electronically as an XML file named “07039-2141WO1.xml.” The XML file, created on Apr. 20, 2023, is 5000 bytes in size. The material in the XML file is hereby incorporated by reference in its entirety.

### TECHNICAL FIELD

This document relates to methods and materials involved in assessing and/or treating mammals having a caveolinopathy disease (e.g., rippling muscle disease (RMD)). For example, this document relates to methods and materials for using a caveolae-associated protein 4 (cavin-4) polypeptide and/or one or more fragments of a cavin-4 polypeptide to detect the presence or absence of autoantibodies present in immune-mediated caveolinopathy disease (e.g., immune-mediated rippling muscle disease (iRMD)). For example, this document relates to methods and materials for using a polypeptide (e.g., an antibody) that binds to a cavin-4 polypeptide to detect the presence or absence a decreased level of a cavin-4 polypeptide in immune-mediated caveolinopathy disease (e.g., iRMD).

### BACKGROUND INFORMATION

RMD is a rare myopathy characterized by abnormal muscle hyperexcitability. RMD is typically electrically silent, exhibiting wave-like muscle contractions (rippling) and percussion/stretch-induced muscle mounding (Schulte-Mattler et al., Neurology, 64 (2): 364-367 (2005)). Responsiveness to immunotherapy in some patients supports an autoimmune pathogenesis suggesting that those patients have iRMD. In contrast to hereditary rippling muscle disease (hRMD), associated to date with pathogenic variants in caveolin-3 (CAV3) or, less frequently, cavin-1 (CAVIN1) genes, patients with iRMD lack a defined genetic defect (Betz et al., Nat. Genet., 28 (3): 218-219 (2001); and Rajab et al., PLOS Genet., 6 (3): e1000874 (2010)). No disease-specific autoantibody biomarker for iRMD has been identified (Lo et al., Neuromusc. Disord., 21 (3): 194-203 (2011); and Walker et al., Bioc. Biophys. Res. Comm., 264 (2): 430-435 (1999)).

### SUMMARY

This document provides methods and materials for assessing and/or treating mammals (e.g., humans) having a caveolinopathy disease (e.g., RMD). For example, this document provides methods and materials for detecting autoantibodies in mammals (e.g., humans) having caveolinopathy disease (e.g., iRMD). As described herein, a specific IgG autoantibody marker is found in serum of some individuals having iRMD (also referred to herein as iRMD-specific autoantibodies). Also as described herein, iRMD-specific autoantibodies can target and bind to a cavin-4 polypeptide. Accordingly, a cavin-4 polypeptide and/or one or more fragments of a cavin-4 polypeptide can be used to determine if a sample (e.g., a serum sample) obtained from a mammal (e.g., a human) having RMD contains iRMD-specific autoantibodies (e.g., anti-cavin-4 polypeptide antibodies), and the presence of iRMD-specific autoantibodies (e.g., anti-cavin-4 polypeptide antibodies) can be used to identify the mammal as having iRMD. Serological detection of iRMD-specific autoantibodies (e.g., anti-cavin-4 polypeptide antibodies) can enable earlier diagnosis of iRMD, and can allow clinicians to provide appropriate treatment. Similarly, a polypeptide (e.g., an antibody) that can bind to a cavin-4 polypeptide can be used to determine if a sample (e.g., a muscle tissue sample) obtained from a mammal (e.g., a human) having RMD contains a decreased level of a cavin-4 polypeptide, and the presence of a decreased level of a cavin-4 polypeptide can be used to identify the mammal as having iRMD.

In general, one aspect of this document features methods for determining whether or not a mammal has an immune-mediated caveolinopathy disease. The methods can include, or consist essentially of, (a) contacting a serum sample from a mammal with a composition comprising an antigen to form an antigen-cavin-4 polypeptide autoantibody complex if the serum sample includes an anti-cavin-4 polypeptide autoantibody, where the antigen is a cavin-4 polypeptide, a fragment of a cavin-4 polypeptide having the ability to bind to the anti-cavin-4 polypeptide autoantibody, or a variant of a cavin-4 polypeptide having the ability to bind to the anti-cavin-4 polypeptide autoantibody; and (b) detecting the presence or absence of the complex, where the presence of the complex indicates that the mammal has the immune-mediated caveolinopathy disease, and where the absence of the complex indicates that the mammal does not have the immune-mediated caveolinopathy disease. The composition can include a cell lysate obtained from a cell (a) including exogenous nucleic acid encoding the antigen and (b) expressing the antigen. The antigen can be the cavin-4 polypeptide. The antigen can be the variant. The antigen can be the fragment. The fragment can consist of the amino acid sequence set forth in any one of SEQ ID NOs: 1-3. The antigen can be covalently linked to a detectable label. The detectable label can be a green fluorescent protein (GFP) polypeptide, tetramethylrhodamine isothiocyanate (TRITC), fluorescein isothiocyanate (FITC), a poly(His) tag, a glutathione-S-transferase (GST) tag, or biotin. The detecting can include an immunological assay. The mammal can be a human. The method can include detecting the presence of the complex and classifying the mammal as having the immune-mediated caveolinopathy disease. The method can include detecting the absence of the complex and classifying the mammal as not having the immune-mediated caveolinopathy disease. The immune-mediated caveolinopathy disease can be iRMD.

In another aspect, this document features methods for determining whether or not a mammal has an immune-mediated caveolinopathy disease. The methods can include, or consist essentially of, (a) detecting the presence or absence a decreased level of a cavin-4 polypeptide in a muscle tissue sample from a mammal; and (b) classifying the mammal as having the immune-mediated caveolinopathy disease if the presence is determined; and (c) classifying the mammal as not having the immune-mediated caveolinopathy disease if the absence is determined. The detecting can include an immunological assay. The mammal can be a human. The muscle tissue sample can be a skeletal muscle sample or a cardiac muscle tissue sample. The method can include detecting the presence of the decreased level and classifying the mammal as having the immune-mediated caveolinopathy disease. The method can include detecting the absence of the decreased level and classifying the mammal as not having the immune-mediated caveolinopathy disease. The immune-mediated caveolinopathy disease can be iRMD.

In another aspect, this document features methods for treating a mammal having an immune-mediated caveolinopathy disease. The methods can include, or consist essentially of, (a) determining that a serum sample from a mammal includes the presence of anti-cavin-4 polypeptide autoantibodies or a muscle tissue sample from the mammal includes the presence of a decreased level of a cavin-4 polypeptide, and (b) administering an immunosuppressant to the mammal. Determining that the serum sample from the mammal includes the presence of anti-cavin-4 polypeptide autoantibodies can include: (i) contacting the serum sample with a composition including an antigen to form an antigen-anti-cavin-4 polypeptide autoantibody complex if the sample comprises an anti-cavin-4 polypeptide autoantibody, where the antigen is a cavin-4 polypeptide, a fragment of a cavin-4 polypeptide having the ability to bind to the anti-cavin-4 polypeptide autoantibody, or a variant of a cavin-4 polypeptide having the ability to bind to the anti-cavin-4 polypeptide autoantibody; and (ii) detecting the presence of the complex, thereby determining that the mammal comprises the presence of said anti-cavin-4 polypeptide autoantibodies. The composition can include a cell lysate obtained from a cell (a) comprising exogenous nucleic acid encoding the antigen and (b) expressing the antigen. The antigen can be the cavin-4 polypeptide. The antigen can be the variant. The antigen can be the fragment. The fragment can consist of the amino acid sequence set forth in any one of SEQ ID NOs: 1-3. The antigen can be covalently linked to a detectable label. The detectable label can be a GFP polypeptide, TRITC, FITC, a poly(His) tag, a GST tag, or biotin. The determining that said the tissue sample from the mammal includes the presence of the decreased level of the cavin-4 polypeptide can include: (i) contacting the muscle tissue sample with a polypeptide having the ability to bind to the cavin-4 polypeptide; and (ii) detecting the presence of the decreased level of the cavin-4 polypeptide, thereby determining that the muscle tissue sample from the mammal includes the presence of the decreased level of the cavin-4 polypeptide. The mammal can be a human. The immune-mediated caveolinopathy disease can be iRMD. The immunosuppressant can be rituximab, mycophenolate, or azathioprine.

In another aspect, this document features methods for treating an immune-mediated caveolinopathy disease. The methods can include, or consist essentially of, administering an immunosuppressant to a mammal that was identified as having a caveolinopathy disease and anti-cavin-4 polypeptide autoantibodies. The identifying can include: (a) contacting a sample from the mammal with a composition including an antigen to form an antigen-anti-cavin-4 polypeptide autoantibody complex if the sample includes an anti-cavin-4 polypeptide autoantibody, where the antigen is a cavin-4 polypeptide, a fragment of a cavin-4 polypeptide having the ability to bind to the anti-cavin-4 polypeptide autoantibody, or a variant of a cavin-4 polypeptide having the ability to bind to the anti-cavin-4 polypeptide autoantibody; and (b) detecting the presence of the complex, thereby identifying the mammal as having the anti-cavin-4 polypeptide autoantibodies. The composition can include a cell lysate obtained from a cell (a) comprising exogenous nucleic acid encoding the antigen and (b) expressing the antigen. The antigen can be the cavin-4 polypeptide. The antigen can be the variant. The antigen can be the fragment. The fragment can consist of the amino acid sequence set forth in any one of SEQ ID NOs: 1-3. The antigen can be covalently linked to a detectable label. The detectable label can be a GFP polypeptide, TRITC, FITC, a poly(His) tag, a GST tag, or biotin. The mammal can be a human. The sample can be a serum sample. The immune-mediated caveolinopathy disease can be iRMD. The immunosuppressant can be rituximab, mycophenolate, or azathioprine.

In another aspect, this document features methods for treating a mammal having a caveolinopathy disease. The methods can include, or consist essentially of, (a) determining that a serum sample from the mammal lacks the presence of anti-cavin-4 polypeptide autoantibodies or that a muscle sample from the mammal lacks a decreased level of a cavin-4 polypeptide, and (b) administering a therapy for the caveolinopathy disease to the mammal, wherein the therapy is not an immunosuppressant. The determining that the serum sample from the mammal lacks the presence of anti-cavin-4 polypeptide autoantibodies can include: (i) contacting the serum sample with a composition including an antigen to form an antigen-anti-cavin-4 polypeptide autoantibody complex if the serum sample includes an anti-cavin-4 polypeptide autoantibody, where the antigen is a cavin-4 polypeptide, a fragment of a cavin-4 polypeptide having the ability to bind to the anti-cavin-4 polypeptide autoantibody, or a variant of a cavin-4 polypeptide having the ability to bind to the anti-cavin-4 polypeptide autoantibody; and (ii) detecting the absence of the complex, thereby that the serum sample from the mammal lacks the presence of anti-cavin-4 polypeptide autoantibodies. The composition can include a cell lysate obtained from a cell (a) comprising exogenous nucleic acid encoding the antigen and (b) expressing the antigen. The antigen can be the cavin-4 polypeptide. The antigen can be the variant. The antigen can be the fragment. The fragment can consist of the amino acid sequence set forth in any one of SEQ ID NOs: 1-3. The antigen can be covalently linked to a detectable label. The detectable label can be a GFP polypeptide, TRITC, FITC, a poly(His) tag, a GST tag, or biotin. The determining that the muscle tissue sample from the mammal lacks the presence of the decreased level of the cavin-4 polypeptide can include: (i) contacting the muscle tissue sample with a polypeptide having the ability to bind to the cavin-4 polypeptide; and (ii) detecting an absence of the decreased level of the cavin-4 polypeptide, thereby determining that the muscle tissue sample from the mammal lacks the presence of the decreased level of the cavin-4 polypeptide. The mammal can be a human. The caveolinopathy disease can be RMD that is not immune-mediated. The therapy for the caveolinopathy disease can be a weight control therapy or physical therapy.

In another aspect, this document features methods for treating a caveolinopathy disease. The methods can include, or consist essentially of, administering a therapy for a caveolinopathy disease to a mammal that was identified as lacking anti-cavin-4 polypeptide autoantibodies, wherein the therapy is not an immunosuppressant. The identifying can include: (a) contacting a sample from the mammal with a composition including an antigen to form an antigen-anti-cavin-4 polypeptide autoantibody complex if the sample includes an anti-cavin-4 polypeptide autoantibody, where the antigen is a cavin-4 polypeptide, a fragment of a cavin-4 polypeptide having the ability to bind to the anti-cavin-4 polypeptide autoantibody, or a variant of a cavin-4 polypeptide having the ability to bind to the anti-cavin-4 polypeptide autoantibody; and (b) detecting the absence of the complex, thereby identifying the mammal as lacking anti-cavin-4 polypeptide autoantibodies. The composition can include a cell lysate obtained from a cell (a) comprising exogenous nucleic acid encoding the antigen and (b) expressing the antigen. The antigen can be the cavin-4 polypeptide. The antigen can be the variant. The antigen can be the fragment. The fragment can consist of the amino acid sequence set forth in any one of SEQ ID NOs: 1-3. The antigen can be covalently linked to a detectable label. The detectable label can be a GFP polypeptide, TRITC, FITC, a poly(His) tag, a GST tag, or biotin. The mammal can be a human. The sample can be a serum sample. The caveolinopathy disease can be RMD that is not immune-mediated. The therapy for the caveolinopathy disease can be a weight control therapy or physical therapy.

In another aspect, this document features methods for treating an immune-mediated caveolinopathy disease. The methods can include, or consist essentially of, (a) removing blood from a mammal having a immune-mediated caveolinopathy disease, thereby obtaining removed blood, (b) removing at least some anti-cavin-4 polypeptide autoantibodies from the removed blood, thereby obtaining processed blood, and (c) reintroducing the processed blood into the mammal. The step (b) can include contacting the removed blood with immobilized antigen, where the antigen is a cavin-4 polypeptide, a fragment of a cavin-4 polypeptide having the ability to bind to an anti-cavin-4 polypeptide autoantibody, or a variant of a cavin-4 polypeptide having the ability to bind to an anti-cavin-4 polypeptide autoantibody. The antigen can be the cavin-4 polypeptide. The antigen can be the variant. The antigen can be the fragment. The fragment can consist of the amino acid sequence set forth in any one of SEQ ID NOs: 1-3. The mammal can be a human.

In another aspect, this document features methods for treating a mammal having an immune-mediated caveolinopathy disease. The methods can include, or consist essentially of, administering to a mammal, a polypeptide comprising (i) a cavin-4 polypeptide, a fragment of a cavin-4 polypeptide having the ability to bind to an anti-cavin-4 polypeptide autoantibody, or a variant of a cavin-4 polypeptide having the ability to bind to an anti-cavin-4 polypeptide autoantibody, and (ii) an IgG1-derived Fc fragment. The IgG1-derived Fc fragment can include a dimeric IgG1-derived Fc fragment. The IgG1-derived Fc fragment can be a human IgG1-derived Fc fragment. The polypeptide can include the cavin-4 polypeptide. The polypeptide can include the variant. The polypeptide can include the fragment. The fragment can consist of the amino acid sequence set forth in any one of SEQ ID NOs: 1-3.

In another aspect, this document features uses of a composition including an antigen to treat an immune-mediated caveolinopathy disease, where the antigen is a cavin-4 polypeptide, a fragment of a cavin-4 polypeptide having the ability to bind to an anti-cavin-4 polypeptide autoantibody, or a variant of a cavin-4 polypeptide having the ability to bind to an anti-cavin-4 polypeptide autoantibody.

In another aspect, this document features uses of a composition including an antigen to treat an immune-mediated caveolinopathy disease, where the antigen is a cavin-4 polypeptide, a fragment of a cavin-4 polypeptide having the ability to bind to an anti-cavin-4 polypeptide autoantibody, or a variant of a cavin-4 polypeptide having the ability to bind to an anti-cavin-4 polypeptide autoantibody, where the use includes removing at least some anti-cavin-4 polypeptide autoantibodies from the blood of a mammal having the immune-mediated caveolinopathy disease.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C: Validation of cavin-4 IgG specificity by transfected cell-based immunofluorescence, western blot and tissue-based immunofluorescence assays. FIG. 1A: tetramethylrhodamine isothiocyanate (TRITC)-conjugated anti-human IgG bound patient IgG demonstrated binding to recombinant cavin-4 (panel A1); GFP-tagged cavin-4 protein expressed on the plasma membrane of transiently transfected COS7 cells (panel A2); merged image (panel A3). FIG. 1B: Western blot of COS7 cell lysate containing recombinant cavin-4 protein demonstrated binding of IgG in 8 of the 10 iRMD patients' sera and a commercial cavin-4-specific rabbit IgG (designated “+”) to a ˜70 kD protein; no healthy control serum IgG (“N”) bound. FIG. 1C: Dual immunostaining of cryosectioned rat skeletal muscle by a commercial cavin-4-specific rabbit IgG (panels C2 and C5) and by a patient IgG (panel C1) and healthy control IgG (panel C4) demonstrated colocalization with patient IgG (panel C3) but not with healthy control IgG (panel C6). Nuclei are stained blue by DAPI.

FIG. 2: Cavin-4 and caveolin-3 muscle immunohistochemistry. Healthy control muscle sections demonstrate uniform sarcolemmal distribution of caveolin-3 (panel A), cavin-4 (panel B), and dystrophin (panel C) immunoreactivities. By comparison to control, muscle from a patient with iRMD displays a mosaic pattern of sarcolemmal immunoreactivities for caveolin-3 (markedly attenuated foci, panel D) and cavin-4 (moderately attenuated, panel E), but normal dystrophin immunoreactivity (panel F). Fibers with attenuated caveolin-3 and cavin-4 immunoreactivities are aligned on sequential sections. Muscle from a patient with hereditary rippling muscle disease (hRMD; CAV3, c.99C>G, p.Asn33Lys) demonstrates diffuse attenuation of sarcolemmal caveolin-3 immunoreactivity (panel G) with preservation of cavin-4 (panel H) and dystrophin (panel I).

FIG. 3: Inflammation, MHC-I upregulation, and complement deposits favor a primary immune-mediated pathogenesis for iRMD. Rectangles enclose fibers with marked attenuation of cavin-4 sarcolemmal immunoreactivity (panel A) and upregulated MHC-I (panel B). Hematoxylin and eosin (H&E)-stained section (panel C) demonstrates inflammatory cells in perimysium and endomysium, and necrotic muscle fibers (arrow). Immunoreactive deposits of complement membrane attack complex (MAC) on the sarcolemma of scattered non-necrotic fibers (area distant from the inflammatory reaction) (panel D).

FIGS. 4A-4C: Discovery of cavin-4 autoantibodies in iRMD patient sera. Immunoprecipitation of human proteome phage display library (1010 plaque-forming units (PFU) per milliliter) by IgG in sera from iRMD patients (IP1-5) and healthy controls (IP6, IP7). FIG. 4A: Cavin-4 protein was bound by IgG in four of the five patients' sera (IP1-4, IP4 was negative when tested by validation methodology, therefore considered seronegative) but not by one patient's serum (IP5, patient P10) nor by healthy control sera. FIG. 4B: Note common enrichment of cavin-4 oligopeptide fragments 5, 9, and 10 by IgG in iRMD patients' sera. FIG. 4C: Peptide sequence (SEQ ID NO:4) of fragments 5, 9, and 10 are bold and underlined, with fragments 9 and 10 overlapping (with the overlapping segment shown in a double underline).

FIG. 5: Reduced cavin-4 expression in seropositive patient muscle biopsies. Western blot demonstrating reduced cavin-4 protein concentration in the muscle biopsies of cavin-4 IgG seropositive patients (P1-6; Lane 1-6), compared to cavin-4 seronegative iRMD cases (Lane 7-8). All patients except for patient 6 (dashed box) had relatively similar GAPDH concentrations supporting equitable muscle protein loading.

### DETAILED DESCRIPTION

This document is based, at least in part, on the discovery that a specific IgG autoantibody marker is found in serum of some individuals having iRMD, and the discovery that a cavin-4 polypeptide is the antigen target of the iRMD-specific autoantibodies.

This document provides methods and materials for assessing and/or treating mammals (e.g., humans) having a caveolinopathy disease (e.g., RMD). For example, this document provides methods and materials for detecting iRMD-specific autoantibodies (e.g., anti-cavin-4 polypeptide antibodies) in mammals (e.g., humans) having RMD. In some cases, a cavin-4 polypeptide and/or one or more fragments of a cavin-4 polypeptide can be used to determine if a sample contains iRMD-specific autoantibodies (e.g., anti-cavin-4 polypeptide antibodies). For example, a sample (e.g., a serum sample) obtained from a mammal (e.g., a human) having RMD can be contacted with one or more fragments of a cavin-4 polypeptide such that an iRMD-specific autoantibody (e.g., an anti-cavin-4 polypeptide antibody), if present, forms a complex with the fragment of the cavin-4 polypeptide (an antibody-cavin-4 fragment complex). In some cases, a sample (e.g., a serum sample) obtained from a mammal (e.g., a human) having RMD can be contacted with cells designed to express a cavin-4 polypeptide and/or a lysate from cells designed to express a cavin-4 polypeptide such that an iRMD-specific autoantibody (e.g., an anti-cavin-4 polypeptide antibody), if present, forms a complex with the cavin-4 polypeptide (an antibody-cavin-4 polypeptide complex). The presence of iRMD-specific autoantibodies (e.g., anti-cavin-4 polypeptide antibodies) can be used to identify a mammal (e.g., a human) as having iRMD. In some cases, a polypeptide (e.g., an antibody) that can bind a cavin-4 polypeptide can be used to determine if a sample contains a decreased level of a cavin-4 polypeptide. For example, a sample (e.g., a muscle tissue sample) obtained from a mammal (e.g., a human) having RMD can be contacted with one or more polypeptides that can bind a cavin-4 polypeptide such that the presence, absence, or level of the cavin-4 polypeptide in the sample can be determined. Also provided herein are materials and methods for treating mammals (e.g., humans) having a caveolinopathy disease (e.g., RMD) based, at least in part, on the presence or absence of anti-cavin-4 polypeptide autoantibodies (e.g., iRMD-specific autoantibodies) in a sample (e.g., a serum sample) and/or the presence or absence of a decreased level of a cavin-4 polypeptide in a sample (e.g., muscle tissue sample) obtained from the mammal.

Any appropriate mammal having a caveolinopathy disease (e.g., RMD) can be assessed and/or treated as described herein. Examples of mammals that can have a caveolinopathy disease (e.g., RMD) and can be assessed and/or treated as described herein include, without limitation, primates (e.g., humans and monkeys), dogs, cats, horses, cows, pigs, sheep, rabbits, mice, rats, and goats. For example, humans having a caveolinopathy disease (e.g., RMD) can be assessed for the presence or absence of anti-cavin-4 polypeptide autoantibodies (iRMD-specific autoantibodies) and, optionally, can be treated as described herein. In some cases, a human that is assessed and/or treated as described herein can be an adult (e.g., can be older than 18 years of age). In some cases, a human that is assessed and/or treated as described herein can be a child (e.g., can be from about 1 year of age to about 18 years of age). For example, a human that is assessed and/or treated as described herein can be an adolescent (e.g., can be from about 10 years of age to about 18 years of age).

A mammal (e.g., human) that can be assessed and/or treated as described herein can have a caveolinopathy disease (e.g., RMD) that affects any one or more muscles within the mammal's body. Examples of muscles that can be affected by RMD include, without limitation, skeletal muscles, smooth muscles and cardiac muscles.

In some cases, a mammal (e.g., human) having RMD can have myasthenia gravis (MG; e.g., ocular MG and generalized MG).

In some cases, a mammal (e.g., human) having RMD can have autoantibodies specific for a muscle acetylcholine receptor (AChR) polypeptide (e.g., can be anti-AChR seropositive).

Any appropriate sample from a mammal (e.g., human) having a caveolinopathy disease (e.g., RMD) can be assessed for the presence or absence of anti-cavin-4 polypeptide autoantibodies (e.g., iRMD-specific autoantibodies) as described herein. Examples of samples that can be assessed as described herein include, without limitation, blood samples (e.g., whole blood samples, serum samples, and plasma samples), CSF samples, urine samples, and tissue samples (e.g., muscle tissue samples such as skeletal muscle tissue samples and cardiac muscle tissue samples). When a sample is a tissue sample, the tissue sample can be a fresh sample (e.g., a frozen fresh sample) or a fixed sample (e.g., a formaldehyde-fixed sample or a formalin-fixed sample). In some cases, one or more biological molecules can be isolated from a sample. For example, polypeptides (e.g., antibodies) can be isolated from a sample and can be assessed as described herein.

Any appropriate cavin-4 polypeptide can be used to detect the presence or absence of anti-cavin-4 polypeptide autoantibodies (e.g., iRMD-specific autoantibodies) in sample (e.g., a sample obtained from a mammal such as a human having a caveolinopathy disease (e.g., RMD)). Examples of cavin-4 polypeptides that can be used to detect anti-cavin-4 polypeptide autoantibodies (e.g., iRMD-specific autoantibodies) include, without limitation, those set forth in the National Center for Biotechnology Information (NCBI) databases at, for example, accession no. NM_001018116.1.

Any appropriate fragment of a cavin-4 polypeptide can be used to detect an anti-cavin-4 polypeptide autoantibodies (e.g., an iRMD-specific autoantibody). A fragment of a cavin-4 polypeptide can be any appropriate length (e.g., can include any number of amino acids) provided that it retains the ability to bind to an anti-cavin-4 polypeptide autoantibody (e.g., an iRMD-specific autoantibody). For example, a fragment of a cavin-4 polypeptide can be from about 4 amino acids in length to about 363 amino acids in length (e.g., from about 4 amino acids in length to about 350 amino acids, from about 4 amino acids in length to about 325 amino acids, from about 4 amino acids in length to about 300 amino acids, from about 4 amino acids in length to about 275 amino acids, from about 4 amino acids in length to about 250 amino acids, from about 4 amino acids in length to about 225 amino acids, from about 4 amino acids in length to about 200 amino acids, from about 4 amino acids in length to about 175 amino acids, from about 4 amino acids in length to about 150 amino acids, from about 4 amino acids in length to about 125 amino acids, from about 4 amino acids in length to about 100 amino acids, from about 4 amino acids in length to about 75 amino acids, from about 4 amino acids in length to about 50 amino acids, from about 4 amino acids in length to about 25 amino acids, from about 10 amino acids in length to about 363 amino acids, from about 25 amino acids in length to about 363 amino acids, from about 50 amino acids in length to about 363 amino acids, from about 75 amino acids in length to about 363 amino acids, from about 100 amino acids in length to about 363 amino acids, from about 125 amino acids in length to about 363 amino acids, from about 150 amino acids in length to about 363 amino acids, from about 175 amino acids in length to about 363 amino acids, from about 200 amino acids in length to about 363 amino acids, from about 225 amino acids in length to about 363 amino acids, from about 250 amino acids in length to about 363 amino acids, from about 275 amino acids in length to about 363 amino acids, from about 300 amino acids in length to about 363 amino acids, from about 325 amino acids in length to about 363 amino acids, from about 350 amino acids in length to about 363 amino acids, from about 50 amino acids in length to about 300 amino acids, from about 75 amino acids in length to about 250 amino acids, from about 100 amino acids in length to about 200 amino acids, from about 10 amino acids in length to about 50 amino acids, from about 50 amino acids in length to about 75 amino acids, from about 75 amino acids in length to about 100 amino acids, from about 100 amino acids in length to about 150 amino acids, from about 150 amino acids in length to about 200 amino acids, from about 200 amino acids in length to about 250 amino acids, or from about 250 amino acids in length to about 300 amino acids in length). In some cases, a fragment of a cavin-4 polypeptide fragment can have an amino acid sequence set forth in any one of SEQ ID NOs: 1-3 (e.g., can consist of an amino acid sequence set forth in any one of SEQ ID NOs: 1-3, can consist essentially of an amino acid sequence set forth in any one of SEQ ID NOs: 1-3, or can comprise an amino acid sequence set forth in any one of SEQ ID NOs: 1-3). In some cases, a fragment of a cavin-4 polypeptide can be a variant of an amino acid sequence set forth in any one of SEQ ID NOs: 1-3. For example, a variant of a fragment of a cavin-4 polypeptide can consist of, consist essentially of, or comprise an amino acid sequence set forth in any one of SEQ ID NOs: 1-3, except that the variant polypeptide includes one, two, three, four, or five amino acid substitutions within the articulated sequence of the sequence identifier (e.g., SEQ ID NO:1, SEQ ID NO:2, or SEQ ID NO:3), has one, two, three, four, or five amino acid residues preceding the articulated sequence of the sequence identifier (e.g., SEQ ID NO:1, SEQ ID NO:2, or SEQ ID NO:3), and/or has one, two, three, four, or five amino acid residues following the articulated sequence of the sequence identifier (e.g., SEQ ID NO:1, SEQ ID NO:2, or SEQ ID NO:3), provided that the fragment of a cavin-4 polypeptide retains the ability to bind to an anti-cavin-4 polypeptide autoantibody (e.g., an iRMD-specific autoantibody). Examples of fragments of a cavin-4 polypeptide that can be used to detect anti-cavin-4 polypeptide autoantibodies (e.g., iRMD-specific autoantibodies) include, without limitation, those fragments of a cavin-4 polypeptide shown in Table 1.

TABLE 1
Examples of fragments of cavin-4 polypeptides
and variants thereof.

| Amino Acid Sequence | SEQ ID NO |
|---|---|
| SLSVVKDRNLTENQEEDDDDIFDPP | 1 |
| VDLSSDEEYYVEESRSARLRKSGKE | |
| PSKEAFKMRSLRKGKDRTVAEGEEC | 2 |
| AREMGVDIIARSESLGPISELYSDE | |
| L | |
| CAREMGVDIIARSESLGPISELYSD | 3 |
| ELSEPEHEAARPVYPPHEGREIPTP | |

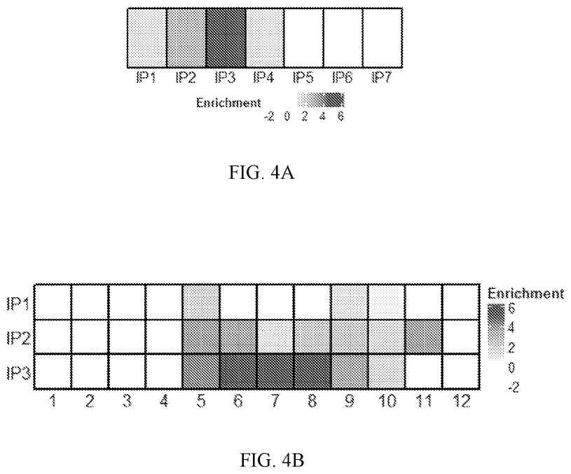

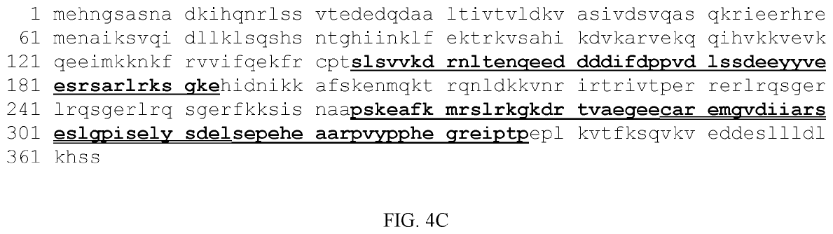

In some cases, fragments of a cavin-4 polypeptide that can be used to detect anti-cavin-4 polypeptide autoantibodies (e.g., iRMD-specific autoantibodies) can be as shown in FIG. 4C.

Any appropriate method can be used to obtain a cavin-4 polypeptide or a fragment of a cavin-4 polypeptide described herein (e.g., a polypeptide that consists of an amino acid sequence set forth in any one of SEQ ID NOs: 1-3, a polypeptide that consists essentially of an amino acid sequence set forth in any one of SEQ ID NOs: 1-3, or a polypeptide that comprises an amino acid sequence set forth in any one of SEQ ID NOs: 1-3) or a variant thereof. In some cases, a cavin-4 polypeptide or a fragment of a cavin-4 polypeptide (or a variant thereof) can be obtained using polypeptide synthesizing methods. For example, a polynucleotide sequence encoding a cavin-4 polypeptide (or a variant thereof) and/or a polynucleotide sequence encoding a fragment of a cavin-4 polypeptide (or a variant thereof) can be inserted into a plasmid or other vector that can then be delivered to host cells that can be induced to transcribe and translate the polynucleotide into the polypeptide. In some cases, a polynucleotide sequence for a larger polypeptide (e.g., a cavin-4 polypeptide) can be inserted into host cells that can produce the larger polypeptide and then that polypeptide can be processed into a smaller polypeptide or a functional variant of interest (e.g., a fragment of a cavin-4 polypeptide).

A cavin-4 polypeptide and/or one or more fragments of a cavin-4 polypeptide (or a variant thereof) can be provided in any appropriate context. In some cases, a cavin-4 polypeptide and/or one or more fragments of a cavin-4 polypeptide (or a variant thereof) that can be used to detect the presence or absence of anti-cavin-4 polypeptide autoantibodies (e.g., iRMD-specific autoantibodies) can be present in a cell lysate. For example, a cavin-4 polypeptide can be present in a cell lysate obtained from a cell having (e.g., designed to have) an exogenous nucleic acid encoding the cavin-4 polypeptide. For example, a fragment of a cavin-4 polypeptide can be present in a cell lysate obtained from a cell having (e.g., designed to have) an exogenous nucleic acid encoding the fragment of a cavin-4 polypeptide.

In some cases, a cavin-4 polypeptide and/or one or more fragments of a cavin-4 polypeptide (or a variant thereof) that can be used to detect the presence or absence of anti-cavin-4 polypeptide autoantibodies (e.g., iRMD-specific autoantibodies) can be present on a cell (e.g., an intact cell). For example, a cavin-4 polypeptide can be present on a cell having (e.g., designed to have) an exogenous nucleic acid encoding the cavin-4 polypeptide and expressing the cavin-4 polypeptide on its surface. For example, a fragment of a cavin-4 polypeptide can be present on a cell having (e.g., designed to have) an exogenous nucleic acid encoding the fragment of a cavin-4 polypeptide and expressing the fragment on its surface.

In some cases, a cavin-4 polypeptide and/or one or more fragments of a cavin-4 polypeptide (or a variant thereof) that can be used to detect the presence or absence of anti-cavin-4 polypeptide autoantibodies (e.g., iRMD-specific autoantibodies) can be substantially pure. The term “substantially pure” as used herein with reference to a cavin-4 polypeptide and/or one or more fragments of a cavin-4 polypeptide (or a variant thereof) refers to material which is substantially or essentially free from components (e.g., other polypeptides, lipids, carbohydrates, and nucleic acid) that normally accompany the material as it is found in its native state. Thus, substantially pure polypeptides as described herein do not contain at least some of the materials normally associated with the polypeptides in their in situ environment. For example, a substantially pure cavin-4 polypeptide and/or one or more substantially pure fragments of a cavin-4 polypeptide (or a variant thereof) can constitute the major component in a mixture of components (e.g., 30% or more, 40% or more, 50% or more, 60% or more, 70% or more, 80% or more, 90% or more, 95% or more, or 99% or more by weight).

In some cases, a cavin-4 polypeptide and/or one or more fragments of a cavin-4 polypeptide (or variant thereof) that can be used to detect the presence or absence of anti-cavin-4 polypeptide autoantibodies (e.g., iRMD-specific autoantibodies) can be present in a composition. A composition including a cavin-4 polypeptide and/or one or more fragments of a cavin-4 polypeptide (or a variant thereof) that can be used to detect the presence or absence of anti-cavin-4 polypeptide autoantibodies (e.g., iRMD-specific autoantibodies) can include any appropriate amount of the cavin-4 polypeptide and/or the one or more fragments of a cavin-4 polypeptide (or a variant thereof). For example, at least 5 percent (e.g., at least 10%, at least 20%, at least 30%, at least 40%, at least 50%, at least 60%, at least 70%, at least 80%, at least 90%, or more) of the polypeptide content of a composition including a cavin-4 polypeptide and/or one or more fragments of a cavin-4 polypeptide (or a variant thereof) can be the cavin-4 polypeptide and/or the one or more fragments of a cavin-4 polypeptide (or the variant thereof). In some cases, a composition including a cavin-4 polypeptide and/or one or more fragments of a cavin-4 polypeptide (or a variant thereof) can be enriched for the cavin-4 polypeptide and/or the one or more fragments of a cavin-4 polypeptide (or the variant thereof).

In some cases, a cavin-4 polypeptide and/or one or more fragments of a cavin-4 polypeptide (or variant thereof) described herein can lack any modification. For example, a cavin-4 polypeptide and/or one or more fragments of a cavin-4 polypeptide (or variant thereof) can be used to detect the presence or absence of anti-cavin-4 polypeptide autoantibodies (e.g., iRMD-specific autoantibodies) without any modification. For example, a cavin-4 polypeptide and/or one or more fragments of a cavin-4 polypeptide (or variant thereof) that can be used to detect the presence or absence of anti-cavin-4 polypeptide autoantibodies (e.g., iRMD-specific autoantibodies) can lack any detectable label.

In some cases, a cavin-4 polypeptide and/or one or more fragments of a cavin-4 polypeptide (or a variant thereof) described herein can be modified. For example, a cavin-4 polypeptide and/or one or more fragments of a cavin-4 polypeptide (or a variant thereof) that can be used for detecting the presence or absence of anti-cavin-4 polypeptide autoantibodies (e.g., iRMD-specific autoantibodies) can include (e.g., can be covalently linked to) one or more labels (e.g., one or more detectable labels). In some cases, a label can be a polypeptide tag (e.g., an affinity tag). In some cases, a label can be a fluorescent label. In some cases, a label can be covalently linked to a chemiluminescent label. In some cases, a label can have enzymatic activity. In some cases, a label can be radioactive. Examples of labels that can be attached to cavin-4 polypeptide or a fragment of a cavin-4 polypeptide (or a variant thereof) include, without limitation, green fluorescent protein (GFP) polypeptides, TRITC, fluorescein isothiocyanate (FITC), poly(His) tag, glutathione-S-transferase (GST) tag, and biotin.

Any appropriate method can be used to detect the presence or absence of anti-cavin-4 polypeptide autoantibodies (e.g., iRMD-specific autoantibodies) in a sample (e.g., a serum sample) obtained from a mammal (e.g., a human) having RMD. For example, immunological assays using a cavin-4 polypeptide and/or one or more fragments of a cavin-4 polypeptide (or a variant thereof) provided herein can be used to determine if a sample contains anti-cavin-4 polypeptide autoantibodies (e.g., iRMD-specific autoantibodies). In some cases, a sample (e.g., a serum sample) obtained from a mammal (e.g., a human) having RMD can be contacted with a cavin-4 polypeptide and/or one or more fragments of a cavin-4 polypeptide (or a variant thereof) described herein such that an anti-cavin-4 polypeptide autoantibody (e.g., an iRMD-specific autoantibody), if present, forms a complex with the cavin-4 polypeptide and/or the one or more fragments of a cavin-4 polypeptide (or the variant thereof), and the presence or absence of a complex can be used to determine whether or not anti-cavin-4 polypeptide autoantibodies (e.g., iRMD-specific autoantibodies) are present in the sample. In some cases, an immobilized cavin-4 polypeptide and/or one or more immobilized fragments of a cavin-4 polypeptide (or a variant thereof) can be used to capture an anti-cavin-4 polypeptide autoantibody (e.g., an iRMD-specific autoantibody) if present within a sample being tested, and an anti-Ig antibody (e.g., an anti-human IgG antibody when testing for human autoantibodies) can be used to determine whether or not anti-cavin-4 polypeptide autoantibodies (e.g., iRMD-specific autoantibodies) were captured. In some cases, an anti-Ig antibody can be labeled (e.g., fluorescently or enzymatically labeled) to aid in detection.

In some cases, the presence or absence of anti-cavin-4 polypeptide autoantibodies (e.g., iRMD-specific autoantibodies) in a sample (e.g., a serum sample) obtained from a mammal (e.g., a human) having RMD can be detected as described in Example 1.

In some cases, methods for assessing a sample (e.g., a serum sample) obtained from a mammal (e.g., a human) having RMD for the presence or absence of anti-cavin-4 polypeptide autoantibodies (e.g., iRMD-specific autoantibodies) as provided herein can be used to identify a mammal as having iRMD. For example, when a mammal (e.g., a human) having RMD is identified as having a presence of anti-cavin-4 polypeptide autoantibodies (e.g., iRMD-specific autoantibodies) in a sample (e.g., a serum sample) obtained from the mammal, the mammal can be classified as having iRMD. For example, when a mammal (e.g., a human) having RMD is identified as lacking anti-cavin-4 polypeptide autoantibodies (e.g., iRMD-specific autoantibodies) in a sample (e.g., a serum sample) obtained from the mammal, the mammal can be classified as not having iRMD (e.g., as having RMD that is not immune-mediated). For example, when a mammal (e.g., a human) having RMD is identified as lacking anti-cavin-4 polypeptide autoantibodies (e.g., iRMD-specific autoantibodies) in a sample (e.g., a serum sample) obtained from the mammal, the mammal can be classified as having RMD that is not immune-mediated.

Any appropriate polypeptide that can bind cavin-4 polypeptide can be used to detect the presence or absence of a decreased level of a cavin-4 polypeptide in sample (e.g., a sample such as a muscle tissue sample obtained from a mammal such as a human having a caveolinopathy disease (e.g., RMD)). The term “decreased level” as used herein with respect to a level of a cavin-4 polypeptide in a sample (e.g., a muscle tissue sample) refers to any level that is lower than a reference level of the polypeptide. The term “reference level” as used herein with respect to a level of a polypeptide refers to the level of the polypeptide typically observed in a control sample. Control samples are samples obtained from mammals that do not have iRMD (e.g., healthy mammals) and/or mammals having immune-mediated diseases that are not associated with anti-cavin-4 polypeptide autoantibodies (e.g., iRMD-specific autoantibodies). It will be appreciated that levels of cavin-4 polypeptides from comparable samples are used when determining whether or not a particular level is a decreased level of a cavin-4 polypeptide. In some cases, a decreased level of a cavin-4 polypeptide can be at least 5% (e.g., about 5%, about 10%, about 20%, about 30%, about 40%, about 50%, about 60%, about 70%, about 80%, about 90%, about 95%, or more) lower than a reference level of a cavin-4 polypeptide. For example, a decreased level of a cavin-4 polypeptide can be a level that is at least 2 (e.g., at least 5, at least 10, at least 15, at least 20, at least 25, at least 35, or at least 50) fold less than a reference level of a cavin-4 polypeptide.

In some cases, a polypeptide that can bind a cavin-4 polypeptide can include an antigen-binding domain that can target a cavin-4 polypeptide. An antigen-binding domain that can target a cavin-4 polypeptide can be any appropriate antigen-binding domain that can target a cavin-4 polypeptide. In some cases, an antigen-binding domain that can target a cavin-4 polypeptide can include an antibody or a fragment thereof that binds to a cavin-4 polypeptide. Examples of antigen-binding domains include, without limitation, an antigen-binding fragment (Fab), a heavy chain variable (VH) domain of an antibody, a light chain variable (VL) domain of an antibody, and a single chain variable fragment (scFv).

Examples of polypeptides that can bind a cavin-4 polypeptide and that can be used to detect the presence or absence of a decreased level of a cavin-4 polypeptide include, without limitation, anti-cavin-4 antibodies (e.g., HPA020973, Sigma-Aldrich (St. Louis, MO)).

Any appropriate method can be used to detect the presence or absence of a decreased level of a cavin-4 polypeptide in a sample (e.g., a muscle tissue sample) obtained from a mammal (e.g., a human) having RMD. In some cases, the presence, absence, or level of a cavin-4 polypeptide can be assessed by detecting and/or quantifying mRNA encoding the cavin-4 polypeptide. Examples of methods that can be used to detect and/or quantify mRNA include, without limitation, RT-PCR techniques (e.g., quantitative RT-PCR techniques). In some cases, the presence, absence, or level of a cavin-4 polypeptide can be assessed by detecting and/or quantifying the cavin-4 polypeptide. Examples of methods that can be used to detect and/or quantify polypeptides include, without limitation, immunohistochemistry (IHC) techniques, mass spectrometry techniques (e.g., proteomics-based mass spectrometry assays or targeted quantification-based mass spectrometry assays), western blotting techniques, and enzyme-linked immunoassays (ELISAs).

In some cases, the presence or absence of a decreased level of a cavin-4 polypeptide in a sample (e.g., a muscle tissue sample) obtained from a mammal (e.g., a human) having RMD can be detected as described in Example 1.

In some cases, methods for assessing a sample (e.g., a muscle tissue sample) obtained from a mammal (e.g., a human) having RMD for the presence or absence of a decreased level of a cavin-4 polypeptide as provided herein can be used to identify a mammal as having iRMD. For example, when a mammal (e.g., a human) having RMD is identified as having a presence of a decreased level of a cavin-4 polypeptide in a sample (e.g., a muscle tissue sample) obtained from the mammal, the mammal can be classified as having iRMD. For example, when a mammal (e.g., a human) having RMD is identified as lacking a decreased level of a cavin-4 polypeptide in a sample (e.g., a muscle tissue sample) obtained from the mammal, the mammal can be classified as not having iRMD (e.g., as having RMD that is not immune-mediated). For example, when a mammal (e.g., a human) having RMD is identified as lacking a decreased level of a cavin-4 polypeptide in a sample (e.g., a muscle tissue sample) obtained from the mammal, the mammal can be classified as having RMD that is not immune-mediated.

This document also provides a cavin-4 polypeptide and/or one or more fragments of a cavin-4 polypeptide (or a variant thereof) in the form of a fusion polypeptide (e.g., a selective degradation (seldeg) molecule). For example, a cavin-4 polypeptide can be fused to an IgG1-derived Fc fragment (e.g., a dimeric, human IgG1-derived Fc fragment) to selectively eliminate anti-cavin-4 polypeptide autoantibodies (e.g., iRMD-specific autoantibodies). A cavin-4 polypeptide and/or one or more fragments of a cavin-4 polypeptide (or a variant thereof) can be fused to any appropriate IgG1-derived Fc fragment. In some cases, a cavin-4 polypeptide and/or one or more fragments of a cavin-4 polypeptide (or a variant thereof) can be fused to an IgG1-derived Fc fragment as described elsewhere (see, e.g., Devanaboyina et al., Nature Communications, 8:15314 (2017); and Sun et al., Molecular Therapy, 29 (3): P1312-1323 (2021)). For example, a fragment of a cavin-4 polypeptide (or a variant thereof) can be fused to a IgG1-derived Fc fragment (e.g., a dimeric, human IgG1-derived Fc fragment) to selectively eliminate anti-cavin-4 polypeptide autoantibodies (e.g., iRMD-specific autoantibodies).

This document also provides methods and materials for treating a mammal (e.g., a human) having a caveolinopathy disease (e.g., RMD), where one or more treatments are selected based on whether the mammal is identified as having an immune-mediated caveolinopathy disease (e.g., an iRMD) as described herein (e.g., based, at least in part, on the presence or absence of anti-cavin-4 polypeptide autoantibodies (e.g., iRMD-specific autoantibodies) in a sample (e.g., a serum sample) obtained from a mammal and/or the presence or absence of a decreased level of a cavin-4 polypeptide in a sample (e.g., a muscle tissue sample) obtained from a mammal). For example, a sample (e.g., a serum sample) obtained from a mammal (e.g., a mammal such as a human having a caveolinopathy disease such as RMD) can be assessed for the presence or absence of anti-cavin-4 polypeptide autoantibodies (e.g., iRMD-specific autoantibodies), and one or more treatments can be selected and, optionally, administered, to the mammal based, at least in part, on whether the presence or absence of anti-cavin-4 polypeptide autoantibodies (e.g., iRMD-specific autoantibodies) is detected. For example, a sample (e.g., a muscle tissue sample) obtained from a mammal (e.g., a mammal such as a human having a caveolinopathy disease such as RMD) can be assessed for the presence or absence of a decreased level of a cavin-4 polypeptide, and one or more treatments can be selected and, optionally, administered, to the mammal based, at least in part, on whether the presence or absence of the decreased level of a cavin-4 polypeptide is detected.

In some cases, a cavin-4 polypeptide and/or one or more fragments of a cavin-4 polypeptide (or a variant thereof) described herein can be used in an apheresis method to treat a mammal (e.g., a human) having a caveolinopathy disease (e.g., iRMD). For example, a cavin-4 polypeptide and/or one or more fragments of a cavin-4 polypeptide (or a variant thereof) described herein can be used in an apheresis for the treatment of a caveolinopathy disease (e.g., iRMD) associated with anti-cavin-4 polypeptide autoantibodies (e.g., iRMD-specific autoantibodies) to remove anti-cavin-4 polypeptide autoantibodies (e.g., iRMD-specific autoantibodies) from the mammal. In some cases, an apheresis method to remove anti-cavin-4 polypeptide autoantibodies (e.g., iRMD-specific autoantibodies) from the blood of a mammal (e.g., a human such as a human having iRMD) can include withdrawing blood from the mammal; contacting the blood with a cavin-4 polypeptide and/or one or more fragments of a cavin-4 polypeptide (or a variant thereof) described herein to remove a substantial portion of anti-cavin-4 polypeptide autoantibodies (e.g., iRMD-specific autoantibodies) from the blood; and returning the blood to the mammal. In some cases, methods and extracorporeal systems for apheresis (i.e., the process of withdrawing blood from an individual, removing components from the blood, and returning the blood, or blood depleted of one or more components, to the individual) can be used as described elsewhere (see, for example, U.S. Pat. Nos. 4,708,713; 5,258,503; 5,386,734; and 6,409,696). As used herein, a “substantial portion” means removing at least 20% (e.g., at least: 20%; 30%; 40%; 50%; 60%; 65%; 70%; 75%; 80%; 85%; 90%; 93%; 95%; 96%; 97%; 98%; 99%; 99.5%; 99.8%; or even 100%) of the anti-cavin-4 polypeptide autoantibodies (e.g., iRMD-specific autoantibodies) that were present in the blood prior to removal.

When a mammal (e.g., a human) having a caveolinopathy disease (e.g., RMD) is identified as having an immune-mediated caveolinopathy disease (e.g., iRMD) as described herein (e.g., based, at least in part, on the presence of anti-cavin-4 polypeptide autoantibodies (e.g., iRMD-specific autoantibodies) in a sample (e.g., a serum sample) obtained from a mammal and/or the presence of a decreased level of a cavin-4 polypeptide in a sample (e.g., a muscle tissue sample) obtained from a mammal), the mammal can be administered, or instructed to self-administer, one or more therapies effective to treat an immune-mediated caveolinopathy disease (e.g., iRMD). In some cases, a therapy that can be effective to treat an immune-mediated caveolinopathy disease (e.g., iRMD) can include plasma exchange therapy (plasmapheresis). In some cases, a therapy that can be effective to treat an immune-mediated caveolinopathy disease (e.g., iRMD) can include intravenous immunoglobulin (IVIG) therapy. In some cases, a therapy that can be effective to treat an immune-mediated caveolinopathy disease (e.g., iRMD) can include administering one or more agents that can be effective to treat an immune-mediated caveolinopathy disease (e.g., iRMD). Examples of agents that can be used to treat a mammal identified as having an immune-mediated caveolinopathy disease (e.g., iRMD) as described herein (e.g., based, at least in part, on the presence of anti-cavin-4 polypeptide autoantibodies (e.g., iRMD-specific autoantibodies) in a sample (e.g., a serum sample) obtained from a mammal and/or the presence of a decreased level of a cavin-4 polypeptide in a sample (e.g., a muscle tissue sample) obtained from a mammal) include, without limitation, seldeg molecules including a cavin-4 polypeptide and/or one or more fragments of a cavin-4 polypeptide (or a variant thereof), immunosuppressants (e.g., rituximab, mycophenolate, and azathioprine), steroids (e.g., corticosteroids such as prednisone and methylprednisolone), anti-inflammatory agents, agents (e.g., monoclonal antibodies) that target a neonatal Fc receptor (FcRn), and complement inhibitors (e.g., classical complement pathway inhibitors such as eculizumab).

When a mammal (e.g., a human) having a caveolinopathy disease (e.g., RMD) is identified as having a caveolinopathy disease (e.g., RMD) that is not immune-mediated as described herein (e.g., based, at least in part, on the absence of anti-cavin-4 polypeptide autoantibodies (e.g., iRMD-specific autoantibodies) in a sample (e.g., a serum sample) obtained from a mammal and/or the absence of a decreased level of a cavin-4 polypeptide in a sample (e.g., a muscle tissue sample) obtained from a mammal), the mammal can be administered, or instructed to self-administer, one or more therapies effective to treat a caveolinopathy disease (e.g., RMD) that is not immune-mediated (e.g., one or more treatments that are not immunosuppressants). Examples of therapies that can be used to treat a mammal identified as having a caveolinopathy disease (e.g., RMD) that is not immune-mediated as described herein (e.g., based, at least in part, on the absence of anti-cavin-4 polypeptide autoantibodies (e.g., iRMD-specific autoantibodies) in a sample (e.g., a serum sample) obtained from a mammal and/or the absence of a decreased level of a cavin-4 polypeptide in a sample (e.g., a muscle tissue sample) obtained from a mammal) include, without limitation, weight control therapies (e.g., to avoid obesity), and physical therapy (e.g., to promote mobility and prevent contractures). In some cases, a mammal (e.g., a human) having a caveolinopathy disease (e.g., RMD) that is identified as having a caveolinopathy disease (e.g., RMD) that is not immune-mediated as described herein (e.g., based, at least in part, on the absence of anti-cavin-4 polypeptide autoantibodies (e.g., iRMD-specific autoantibodies) in a sample (e.g., a serum sample) obtained from the mammal and/or the absence of a decreased level of a cavin-4 polypeptide in a sample (e.g., a muscle tissue sample) obtained from the mammal is not administered any immunotherapy.

In some cases, when treating a mammal (e.g., a human) having a caveolinopathy disease (e.g., RMD or iRMD) as described herein, the treatment can be effective to reduce or eliminate one or more symptoms of the caveolinopathy disease RMD or iRMD. Examples of symptoms of RMD include, without limitation, repetitive tensing of one or more muscles, bunching up of one or more muscles, visible rippling of one or more muscles, muscle weakness, muscle fatigue, muscle cramps, muscle stiffness, dysphagia, dysarthria, and diplopia. In some cases, a symptom of RMD can be as described elsewhere (see, e.g., Vorgerd et al., Neurology, 52 (7): 1453-1459 (1999)). For example, the methods and materials described herein can be used to reduce one or more symptoms within a mammal having a caveolinopathy disease (e.g., RMD or iRMD) by, for example, 10, 20, 30, 40, 50, 60, 70, 80, 90, 95, or more percent.

In some cases, when treating a mammal (e.g., a human) having an immune-mediated caveolinopathy disease (e.g., iRMD) as described herein, the treatment can be effective to reduce or eliminate inflammation within the mammal (e.g., within one or more of the mammal's muscles). For example, the methods and materials described herein can be used to reduce inflammation within a mammal having RMD (e.g., iRMD) by, for example, 10, 20, 30, 40, 50, 60, 70, 80, 90, 95, or more percent.

In some cases, when treating a mammal (e.g., a human) an immune-mediated caveolinopathy disease (e.g., iRMD) as described herein, the treatment can be effective to reduce or eliminate the number of anti-cavin-4 polypeptide autoantibodies (e.g., iRMD-specific autoantibodies) within the mammal. For example, the number of anti-cavin-4 polypeptide autoantibodies (e.g., iRMD-specific autoantibodies) present within a mammal can be reduced using the methods and materials described herein. In some cases, the methods and materials described herein can be used to reduce the number of anti-cavin-4 polypeptide autoantibodies (e.g., iRMD-specific autoantibodies) present within a mammal having an immune-mediated caveolinopathy disease (e.g., an iRMD) by, for example, 10, 20, 30, 40, 50, 60, 70, 80, 90, 95, or more percent. In some cases, the number of anti-cavin-4 polypeptide autoantibodies (e.g., iRMD-specific autoantibodies) present within a mammal does not increase.

In some cases, methods for assessing a sample (e.g., a serum sample) obtained from a mammal (e.g., a human) having a caveolinopathy disease (e.g., RMD or iRMD) can be used to monitor a course of treatment. In some cases, a mammal being treated for a caveolinopathy disease (e.g., RMD) and identified as having an immune-mediated caveolinopathy disease (e.g., an iRMD) as described herein (e.g., based, at least in part, on the presence of anti-cavin-4 polypeptide autoantibodies (e.g., iRMD-specific autoantibodies) in a sample obtained from a mammal) can be monitored for the presence, absence, or level of anti-cavin-4 polypeptide autoantibodies (e.g., iRMD-specific autoantibodies) to determine whether or not the immune-mediated caveolinopathy disease (e.g., iRMD) is being treated. For example, if the level of anti-cavin-4 polypeptide autoantibodies (e.g., iRMD-specific autoantibodies) present within a mammal being treated as described herein is reduced following the administration of one or more immunosuppressants, the immune-mediated caveolinopathy disease (e.g., iRMD) is being treated. For example, if the level of anti-cavin-4 polypeptide autoantibodies (e.g., iRMD-specific autoantibodies) present within a mammal being treated as described herein is not reduced following the administration of one or more immunosuppressants, the immune-mediated caveolinopathy disease (e.g., iRMD) is not being treated.

In some cases, a cavin-4 polypeptide and/or one or more fragments of a cavin-4 polypeptide (or variant thereof) can be used to induce antigen-specific tolerance (e.g., cavin-4 polypeptide-specific tolerance) in a mammal (e.g., a human). For example, a mammal (e.g., a human) can be administered, or instructed to self-administer, a cavin-4 polypeptide and/or one or more fragments of a cavin-4 polypeptide (or a variant thereof) provided herein to induce cavin-4 polypeptide-specific tolerance in that mammal.

The invention will be further described in the following examples, which do not limit the scope of the invention described in the claims.

### EXAMPLES

### Example 1: Caveolae-Associated Protein (cavin)-4 Autoantibodies in Immune-Mediated Rippling Muscle Disease (iRMD)

This Example describes the identification of autoantibodies to a cavin-4 polypeptide in iRMD patients, and demonstrates that seropositivity for cavin-4 IgG can be used as a serological biomarker for iRMD.

### Patients and Methods

### Standard Protocol Approvals, Registrations, and Patient Selection

Sera from 10 patients with clinical diagnosis of iRMD (Schulte-Mattler et al., Neurology, 64 (2): 364-367 (2005)) were evaluated. All ten patients were evaluated by a neuromuscular specialist to have symptoms and signs of rippling muscle disease, specifically, percussion and/or stretch-induced rolling movements across a muscle or muscle group (rippling) and percussion-induced rapid muscle contraction with or without percussion-induced muscle mounding, without associated spontaneous activity and motor unit action potentials detected by electromyography (electrically silent). An immune-mediated etiology was supported by one or more of the following: mosaic pattern of sarcolemmal caveolin-3 immunoreactivity on muscle biopsy, immunotherapy-responsiveness, and lack of mutations in CAV3 and CAVIN1 genes. Nine had undergone diagnostic muscle biopsy. Caveolin-3 immunohistochemical study was performed on 8 biopsies, 7 of which revealed a mosaic pattern of sarcolemmal caveolin-3 immunoreactivity typical of iRMD. The CAV3 gene was sequenced in 9 cases, and CAVIN1 gene was additionally sequenced in 3 cases. No potentially pathogenic variants were found in these genes. One patient (P10) who lacked the mosaic caveolin-3 pattern was included because of a lack of mutations in both CAV3 and CAVIN1. One patient (P7) with rippling muscle disease, co-existing ocular MG and anti-AChR seropositivity, has an unknown genotype (CAV3 and CAVIN1) and muscle biopsy not performed; however, associated seropositive MG at rippling onset, commonly described in iRMD literature, supported an immune-mediated etiology.

### Phage Immunoprecipitation Sequencing

iRMD patient and control sera were incubated with 1010 plaque-forming units (PFU) per milliliter of the whole human proteome phage-display library, and antibody-bound phage particles were isolated by protein G (Dynabeads; Invitrogen, Carlsbad, CA; Thermo Fisher Scientific, Waltham, MA) immunoprecipitation (DD, AK). Patient IgG bound phage particles were eluted, and next-generation sequencing libraries were prepared using the Illlumina TruSeq Nano DNA library preparation kit with associated indexes. Prepared libraries were sequenced on the Illumina NovaSeq platform utilizing an SP flow cell (Illumina, San Diego, CA). Sequenced reads were processed using an in-house developed bioinformatics pipeline (SD) to identify the putative autoantigen.

### Confirmation of Putative Autoantigen

A putative novel antigen was validated by testing patient sera using a protein expression vector-transfected COS7 cell-based assay (CBA), western blotting, human muscle lysate immunoprecipitation, indirect immunofluorescence on cryosectioned rat skeletal muscle, or all of these methods.

### Control Sera

Archived sera from 124 disease controls (dermatomyositis, 20; immune-mediated necrotizing myopathy, 22; MG without evidence/report of muscle rippling, 56; aquaporin-4 positive neuromyelitis optica, 20; peripheral nerve hyperexcitability, 3; sporadic late onset nemaline myopathy, 3) and 123 healthy controls were tested for putative autoantigen by COS7 CBA.

### Muscle Biopsy Histochemical and Immunohistochemical Studies

Nine of the 10 patients had a muscle biopsy and conventional histochemical studies. Eight patients' biopsies were immunostained for caveolin-3 for diagnostic purposes. Additional immunohistochemical studies were performed on residual biopsy tissue for patients P1-P6 and P9-10. No residual tissue was available for P8. Immunohistochemical analysis was done in comparison to healthy control muscle biopsies and two patients with hRMD due to CAV3 mutations (c.99C>G, p.Asn33Lys; c.169G>A, p. Val57Met). All muscle biopsy slides were analyzed independently.

### Phage Immunoprecipitation Sequencing Bioinformatics

The bioinformatics method started by quality assessing the raw read data of each sample using FASTQC software. Reads were then mapped against a custom-built reference containing the 150 bp-length oligo sequences, each representing a peptide, using bwa-mem software configured with default parameters. A program written in AWK programming language processed the aligned data and enumerated the total number of reads that perfectly mapped (i.e., full length match with no sequence modifications) to each peptide. Sample-wise peptides and their respective read counts were loaded into R programming environment (version 4.0.1) to generate enrichment scores. This process started by generating the mean read count observed for each peptide in control samples. A peptide enrichment score was computed for each (sample, peptide) pair by taking the ratio of counts observed in that pair to the mean count observed in control samples. A protein level enrichment score was computed by summing the enrichment scores observed for all of its peptide hits across all patient samples. Proteins were then ranked by the descending order of their enrichment scores and top 20 proteins were considered for further evaluation.

### Cavin-4 Cell-Based Assay (CBA)

Autoantigen specificity was confirmed by indirect immunofluorescence on COS7 cells transfected with plasmid expressing green fluorescent protein (GFP)-tagged human cavin-4. Cells were fixed (4% paraformaldehyde in PBS, 15 minutes), permeabilized (0.5% Triton X-100 in PBS, 2 minutes), and blocked for 1 hour with normal goat serum (10% in PBS). After PBS wash, cells were incubated for 40 minutes with patient serum (1:200 dilution), or rabbit cavin-4 specific IgG (1:200). After PBS wash and incubation with secondary antibodies (1:200 TRITC-conjugated goat anti-rabbit IgG and goat anti-human IgG; SouthernBiotech, Birmingham, AL). Coverslips were mounted using ProLong Gold antifade medium (containing 4,6-diamidino-2-phenylindole (DAPI); Molecular Probes, Thermo Fisher Scientific). All assays were scored by at least two independent reviewers (DD, MBH). Confocal images were captured using a microscope (×20 or ×40 water immersion lens, LSM710; Carl Zeiss, Oberkochen, Germany). Patients seropositive for cavin-4 IgG were also tested for cavin-4 IgG subclasses using subclass-specific secondary antibodies (mouse anti-human IgG1-4 Fc-specific FITC-conjugated (Southern Biotech)).

### Cavin-4 Immunofluorescence Assay (IFA)

Patient serum and commercial rabbit antibodies were tested on a cryosectioned composite of adult rat skeletal muscle sections. Sections were fixed using 4% paraformaldehyde for 1 minute, permeabilized with 3-([3-cholamidopropyl] dimethylammonio)-1-propanesulfonate, 0.5%, in PBS for 1 minute, and then blocked for 1 hour with normal goat serum (10% in PBS). After PBS rinse, patient specimen (diluted 1:240) combined with commercial cavin-4 IgG (1:100, Invitrogen) was applied. After 40 minutes, and PBS wash, a human-specific secondary antibody conjugated with FITC (Scimedx Corporation, Denville, NJ), combined with a rabbit-specific secondary antibody conjugated with TRITC (1:200, Southern Biotech) was applied. Cover slips were mounted using ProLong Gold antifade medium (containing DAPI; Molecular Probes, Thermo Fisher Scientific). Confocal images were captured using a microscope (×20 or ×40 water immersion lens, LSM710; Carl Zeiss).

### Western Blot

Forty hours after transfection with cavin-4-GFP plasmid, cells were washed once in cold 1×PBS and one mL of cold RIPA (50 mM TrisHCL pH 7.5, 150 mM NaCL, 1.0% Triton-X, 0.1% SDS, Roche protease inhibitor tablet) was added. Cells were scrapped off the dish, lysates were homogenized and allowed to lyse in RIPA for an additional 30 minutes at 4 degrees with gentle agitation. Lysates was used for Western blotting (separated in 10% polyacrylamide; transblotted to nitrocellulose membranes), IgG probes were from healthy controls (1:200), candidate patients (1:200), or commercial cavin-4 specific IgG (1:1000).

### Caveolin-3 Cell-Based Assay

COS7 cells transfected with green fluorescent protein (GFP)-tagged human with caveolin-3-expressing plasmid were fixed (4% paraformaldehyde, 15 minutes), permeabilized (0.5% Triton X-100, 2 minutes), and blocked for 1 hour with normal goat serum (10% in PBS). After PBS wash, cells were incubated for 40 minutes with patient serum (1:200 dilution), or rabbit caveolin-3 specific IgG (1:200). After PBS wash and incubation with secondary antibodies (1:200 TRITC-conjugated goat antirabbit IgG and goat antihuman IgG; SouthernBiotech, Birmingham, AL). Coverslips were mounted using ProLong Gold antifade medium (containing DAPI; Molecular Probes, Thermo Fisher Scientific). Assay were scored by at least two independent reviewers (DD, AD).

### Titin Immunoblots

EUROLINE PNS 12 Ag (Euroimmun) was utilized to evaluate for titin seropositivity. According to the manufacturer's instructions, samples were considered positive when presenting intensity was equal or above 15. All iRMD patients with available sera (all patients, except P8) were tested for the titin IgG (positive results reported in Table 1). 18 additional cavin-IgG negative and striational antibody myasthenia gravis patients were tested, 16 of whom were positive for titin IgG.

TABLE 1
Clinical, laboratory, and muscle pathological features of patients with immune-mediated rippling muscle disease.

| Patient | P1 | P2 | P3 | P4 | P5 |
|---|---|---|---|---|---|
| Symptom onset age (yr) | Mid-70s | Late teens | Mid-50s | Mid-40s | Mid-40s |
| Sex | Female | Male | Male | Male | Male |
| Race | Caucasian | African American | Caucasian | Caucasian | Caucasian |
| Presenting features^{#} | Rippling | Diffuse rippling, | Myalgia, fatigue | Rippling | Diffuse rippling, |
| | quadriceps, | percussion- | | quadriceps, | myalgia, fatigue |
| | myalgia, proximal | induced muscle | | myalgia, fatigue | |
| | weakness,^{a} fatigue | mounding, | | | |
| | | myalgia | | | |
| Follow-up (mo) | 16 | 24 | 6 | 2 | 19 |
| Comorbidities at | Hashimoto | None | Type 2 diabetes | None | None |
| presentation | thyroiditis | | mellitus | | |
| Concurrent MG | Yes | No | No | No | No |
| Cancer; screening | No; FDG-PET/CT | No; none | No; CT chest, | No; none | No; CT chest, |
| performed | body | | abdomen, pelvis | | abdomen, pelvis; |
| | | | | | Testicular US; |
| | | | | | PSA |
| Thymoma | No | No | No | No | No |
| CAV3 sequencing | Normal | Normal | Normal | Normal | Normal |
| CAVIN1 sequencing | NP | Normal | NP | NP | NP |
| CAVIN-4 IgG | Positive | Positive | Positive | Positive | Positive |
| | (≥1:6400) | (≥1:6400) | (1:6400) | (1:1600) | (≥1:6400) |
| CAVIN-4 IgG1 | Positive | Positive | Positive | Positive | Positive |
| CK (IU/L) | 885 | 2625 | 132 | 336 | 374 |
| Muscle AChR IgG | 5.94 ^{b} | Negative | Negative | Negative | Negative |
| (nmol/L serum) | | | | | |
| Other serum | Titin, ANA, | None | Titin, striational | Titin, striational | IgG lambda |
| autoantibodies or IgG | (titer 1280) | | (titer, 7680) | (titer, 480) | MGUS |
| (value) | | | | | (1.1 g/dL) |

Myopathological findings

| | | | | | |
|---|---|---|---|---|---|
| Muscle biopsied | Vastus lateralis | Vastus lateralis | Biceps brachii | Vastus lateralis | Vastus lateralis |
| Inflammation, location | None | None | Rare^{c}, perimysial | None | None |
| | | | perivascular | | |
| Sarcolemmal cavin-4 | Mosaic | Mosaic | Mosaic | Mosaic | Mosaic |
| immunoreactivity | | | | | |
| pattern | | | | | |
| Fibers lacking | 63% | 74% | 84% | 91% | 89% |
| sarcolemmal cavin-4/ | | | | | |
| 10x LPF | | | | | |
| Sarcolemmal caveolin- | Mosaic | Mosaic | Mosaic | Mosaic | Mosaic |
| 3 immunoreactivity | | | | | |
| pattern | | | | | |
| Congruent Cavin-4 | Yes | Yes | Yes | Yes | Yes |
| and caveolin-3 | | | | | |
| immunoreactivities | | | | | |
| MHC-I upregulation | +++^{e} | +^{f} | +^{f} | ++^{g} | +^{f} |
| Sarcolemmal | ++^{g} | ++^{g} | ++^{g} | +^{f} | +^{f} |
| MAC deposition | | | | | |

| Patient | P6 | P7 | P8 | P9 | P10 |
|---|---|---|---|---|---|
| Symptom onset age (yr) | Mid-70s | Mid-60s | Mid-40s | Early 50s | Early 60s |
| Sex | Female | Male | Male | Male | Female |
| Race | Caucasian | Caucasian | Caucasian | Caucasian | Caucasian |
| Presenting features^{#} | Rippling | Rippling in | Rippling calves, | Myalgia, fatigue | Myalgia, |
| | quadriceps, | lower limb | proximal | | proximal |
| | myalgia, | muscles, | weakness,^{a} | | weakness,^{a} |
| | proximal | fatigue | fatigue | | fatigue |
| | weakness,^{a} | | | | |
| | fatigue | | | | |
| Follow-up (mo) | 22 | 1 | 29 | 30 | 6 |
| Comorbidities at | Pulmonary | None | None | None | Raynaud |
| presentation | sarcoidosis, | | | | phenomenon |
| | pernicious | | | | |
| | anemia | | | | |
| Concurrent MG | No | Yes | Yes | No | No |
| Cancer; screening | Breast | No; CT chest | No; CT chest | No; CT chest, | No; CT chest, |
| performed | carcinoma; CT | | | abdomen, pelvis | FDG-PET/CT |
| | chest abdomen, | | | | body |
| | pelvis | | | | |
| Thymoma | No | No | No | No | No |
| CAV3 sequencing | Normal | Normal | NP | Normal | Normal |
| CAVIN1 sequencing | NP | NP | NP | Normal | Normal |
| CAVIN-4 IgG | Positive | Positive | Positive | Negative | Negative |
| | (1:3200) | (1:3200) | (≥1:6400) | | |
| CAVIN-4 IgG1 | Positive | Positive | Positive | Negative | Negative |
| CK (IU/L) | 512 | Unknown | 534 | 566 | 1184 |
| Muscle AChR IgG | 0.12 | 2.89 ^{b} | 6.31 ^{b} | Negative | Negative |
| (nmol/L serum) | | | | | |
| Other serum | Titin, striational | Titin, striational | None | Titin, IgG kappa | P/Q-type VGCC |
| autoantibodies or IgG | (titer, 61440) | (titer, 30720) | | MGUS | (0.16 nmol/L)^{h} |
| (value) | | | | (0.7 g/dL) | |

Myopathological findings

| | | | | | |
|---|---|---|---|---|---|
| Muscle biopsied | Biceps brachii | NP | Vastus lateralis | Triceps | Supraspinatus |
| Inflammation, location | Mild^{d}, | NP | None | None | None |
| | endomysial & | | | | |
| | perimysial/ | | | | |
| | perivascular | | | | |
| Sarcolemmal cavin-4 | Mosaic | NP | NP | Mosaic | Normal |
| immunoreactivity | | | | | |
| pattern | | | | | |
| Fibers lacking | 55% | NP | NP | 18% | 0% |
| sarcolemmal cavin-4/ | | | | | |
| 10x LPF | | | | | |
| Sarcolemmal caveolin- | Mosaic | NP | NP | Mosaic | Normal |
| 3 immunoreactivity | | | | | |
| pattern | | | | | |
| Congruent Cavin-4 | Yes | NP | NP | Yes | NA |
| and caveolin-3 | | | | | |
| immunoreactivities | | | | | |
| MHC-I upregulation | +++^{e} | NP | NP | +^{f} | +^{f} |
| Sarcolemmal | +++^{e} | NP | NP | ++^{g} | ++^{g} |
| MAC deposition | | | | | |

^{#}All patients developed muscle rippling
^{a}Medical Research Council (MRC) grade 4/5 in weak muscles
^{b} Disorder of neuromuscular transmission confirmed by electrodiagnostic testing (>10% decrement on repetitive nerve stimulation and/or abnormal single-fiber electromyography)
^{c}Minimal scattered inflammation
^{d}1 small collection per 5x-power field
^{e}≥2 positive fibers per 10x-power field
^{f}More than 3 positive fibers per biopsy, less than 1 positive fiber per 10X-power field
^{g}1 or more positive fibers per 10X-power field, less than 2 positive fibers per 10X-power field
^{h}No evidence of a pre- or post-synaptic defect of neuromuscular transmission by repetitive nerve stimulation and single-fiber EMG
Ab, antibodies; AChR, acetylcholine receptor; ANA, antinuclear antibody; CK, creatine kinase; LPF, low-power field; MAC, membrane attack complex; MHC-I, major histocompatibility complex I; mo., months; MG, myasthenia gravis; MGUS, monoclonal gammopathy of uncertain significance; NA, not applicable; NP, not performed; VGCC, voltage-gated calcium channel; y, years.

### Human Muscle Lysate Immunoprecipitation Sequencing

For immunoprecipitation, 1 μL of patient's IgG and control normal sera IgG was exposed to 1 mL of human muscle lysate preparation for 1 hour then separately complexed to 10 μL protein G magnetic beads (Dynabeads; Invitrogen, Carlsbad, CA; Thermo Fisher Scientific, Waltham, MA) for 30 minutes (DD, MBH, AK). After washing, beads were boiled for 10 minutes in 2× sample buffer. Eluted sample was electrophoresed using a 5 to 15% polyacrylamide gel, and proteins were located by silver staining (Bio-Rad Laboratories, Hercules, CA) and by Western blot. The immunoreactive bands specific to patient IgG immunoprecipitation was excised from the stained gel, reduced, alkylated with iodoacetamide, and digested with trypsin. Peptides in the digested sample were analyzed using nano high-pressure liquid chromatography electrospray tandem mass spectrometry (Mayo Clinic Medical Genome Facility, Proteomics Core).

### Muscle Biopsy Histochemical and Immunohistochemical Studies

Conventional histochemical studies were performed on fresh frozen muscle biopsy 10-μm thick sections and stained for hematoxylin-eosin, modified Gomori trichrome, NADH dehydrogenase, succinate dehydrogenase, cytochrome c oxidase, acid phosphatase, adenosine triphosphatase (ATPase; at pH 4.3, 4.6, and 9.4), toluidine blue ATPase, phosphorylase, periodic acid-Schiff, oil red O, non-specific esterase, and Congo red. For immunohistochemical studies, muscle sections were fixed in −20° C. acetone for 10 minutes. Non-specific binding was blocked by pre-incubation with 2% PBS-BSA containing 10% donkey (for cavin-4 immunostained sections) or goat serum (for caveolin-3 and dystrophin immunostained sections). For immunohistochemical studies, 10-micron thick consecutive frozen sections were reacted with monoclonal IgGs specific for caveolin-3 (610421, BD Transduction Laboratories, San Diego, CA), cavin-4 (HPA020973, Sigma-Aldrich, St. Louis, MO), dystrophin C-terminal (control protein, NCL-DYS2, Novocastra, Leica Biosystems, Buffalo Grove, IL), major histocompatibility complex I (MHC-I; M073601-2, Agilent Technologies, Santa Clara, CA) and membrane attack complex (MAC (mouse anti-human C5b-9); M077701-5, Agilent Technologies, Santa Clara, CA). Cavin-4, caveolin-3, and dystrophin C-terminal proteins were localized with the immunoperoxidase method, using a biotinylated secondary antibody and the ABC kit (PK-6100; Vector Laboratories, Burlingame, CA) and diaminobenzidine (DAB) working solution (K3468; Dako, Carpinterin, CA). Muscle section incubation with primary antibodies was performed overnight at 4° C., and subsequently with biotinylated secondary antibodies for 45 minutes at room temperature. For MHC-I immunohistochemical studies, sections underwent protein blocking (RE7150-K; NovoLink Polymer Detection System, Novocastra, Leica Biosystems, Buffalo Grove, IL) for 5 minutes followed by PBS rinse for 5 minutes twice and subsequent incubation with the primary MHC-I antibody for 60 minutes. Then followed the suggested procedure of NovoLink Polymer Detection System to finalize immunostaining.

All muscle biopsies were assessed the following parameters: presence, size, and distribution of inflammatory exudate; pattern of caveolin-3 and cavin-4 sarcolemmal immunoreactivity (normal, mosaic, or absent), percentage of fibers lacking caveolin-3 and cavin-4 sarcolemmal immunoreactivity per low-power field (LPF, 10× magnification, averaged across four randomly selected LPFs), caveolin-3 sarcolemmal immunoreactivity pattern matching cavin-4 immunoreactivity. Number of fibers per LPF displaying sarcolemmal MHC-I immunoreactivity and number of non-necrotic fibers displaying sarcolemmal MAC immunoreactivity.

### Patient Muscle Western Blot

Frozen patient muscle tissue biopsy was washed once in cold 1×PBS and 400 μL of cold RIPA (50 mM TrisHCL pH 7.5, 150 mM NaCL, 1.0% Triton-X, 0.1% SDS, Roche protease inhibitor tablet) was added. The thawed tissue was homogenized periodically over 30 minutes while maintained on ice. Protein concentrations were obtained using BCA protein assay (Pierce, Thermo Scientific). For each muscle sample, 2 lanes, each containing 10 μg of the protein in SDS PAGE sample buffer with β-mercaptoethanol were run on a 4-15% SDS-PAGE gel, followed by transfer of the protein to nitrocellulose paper. Each muscle sample was then subjected to rabbit polyclonal anti-MURC/cavin-4 antibody (HPA020973, Sigma-Aldrich) at a concentration of 1:1000 on one lane and rabbit polyclonal antibody to GAPDH at a concentration of 1:2500 on the other lane, followed by goat HRP-conjugated anti-rabbit secondary antibodies (1:2000).

### Results

### Autoantigen Identification

Seeking a putative autoantigen for iRMD, sera from three patients (P2, P6 and P8) were initially screened by whole human proteome phage immunoprecipitation sequencing (PhIP-Seq) and candidate antigens were identified by using bioinformatics pipeline to process individual Phage-Seq enrichment data. IgG in all three cases bound to peptides corresponding to different regions of the cavin-4 protein (FIG. 4), with three patients having common peptide hits (fragments 5, 9, and 10). Sera from two iRMD patient (P3, P7) were used for subsequent protein G magnetic bead capture and mass spectrometry analysis of a human muscle lysate preparation.

### Clinical Specificity of Cavin-4-IgG

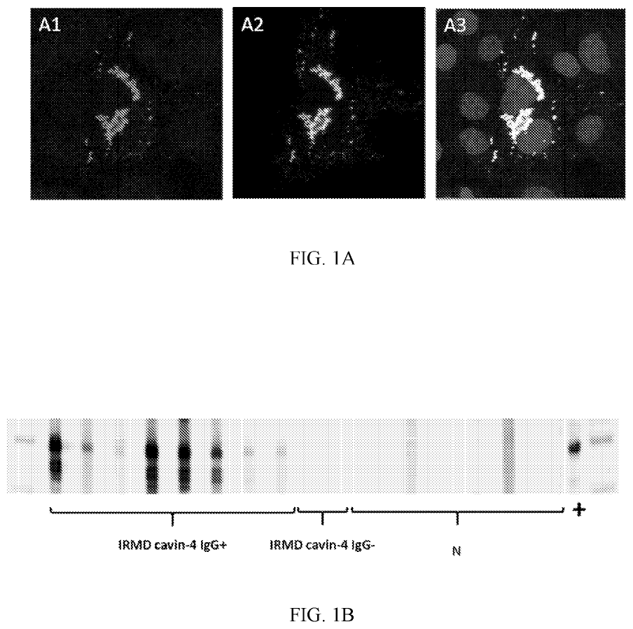

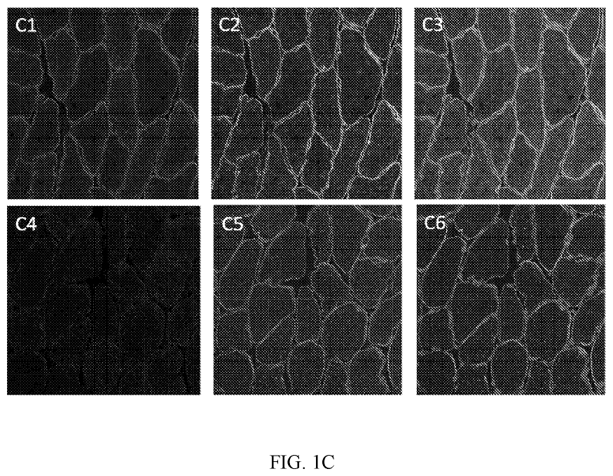

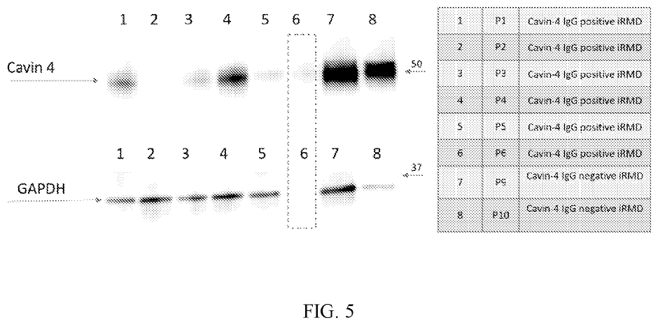

All 10 iRMD patients' sera were tested for cavin-4 IgG by immunofluorescent CBA using cavin-4-transfected COS7 cells as substrate. Eight of the ten iRMD cases (P1-P8) were positive (FIG. 1). IgG in seven of the eight positive sera (P1-P4, P6-P8) colocalized with a commercial cavin-4-specific IgG on rat skeletal muscle by immunostaining (FIG. 1, FIG. 5). Furthermore, IgG in all eight patients' sera yielded a positive band by western blot on lysate containing recombinant cavin-4. IgG in two sera that were negative by CBA on transfected COS7 cell and western blot on denatured cavin-4 protein (P9, P10) were screened by whole human proteome PhIP seq. IgG in one of those sera (P9) bound selectively to Cavin-4 (with lower enrichment score than the initial three index sera) but the other serum (patient P10) was negative (Table 2).

TABLE 2
Summary of methods by which autoantibodies
to cavin-4 were identified in each patient.

| | Patient | Cell-Based | Overexpression | Rodent |
|---|---|---|---|---|
| | Phage | Overexpression | Cell Lysate | Skeletal muscle |
| Patient | Display | Assay | Western blot | Immunohistochemistry |
| 1 | Not tested | Positive | Positive | Positive |
| 2 | Positive | Positive | Positive | Positive |
| 3 | Not tested | Positive | Positive | Positive |
| 4 | Not tested | Positive | Positive | Positive |
| 5 | Not tested | Positive | Positive | Negative |
| 6 | Positive | Positive | Positive | Positive |
| 7 | Not tested | Positive | Positive | Positive |
| 8 | Positive | Positive | Positive | Positive |
| 9 | Positive | Negative | Negative | Negative |
| 10 | Negative | Negative | Negative | Negative |

The cavin-4-reactive IgG in all 8 positive sera was of IgG1 subclass. None of the disease control sera (98 immune-mediated myopathy/neuromuscular junction disorders, 20 autoimmune CNS diseases and 123 healthy subjects) contained cavin-4-reactive IgG. Furthermore, none of the five iRMD patients' sera tested through PhIP Seq contained IgG reactive with caveolin-3 or cavin-1, and none of the 10 iRMD patients' sera tested by CBA were positive for caveolin-3 IgG (Table 3).

TABLE 3
Immune-mediated rippling muscle disease and
disease controls tested on cavin-4 CBA.

| | Cavin-4 CBA |
|---|---|
| Samples tested | positive/total |
| Immune-mediated rippling muscle disease | 8/10 (serum) |
| Dermatomyositis | 0/20 (scrum) |
| Immune-mediated necrotizing myopathy | 0/22 (serum) |
| MG without evidence/report of muscle rippling | 0/56 (serum) |
| Aquaporin-4 positive NMO | 0/20 (serum) |
| Cases with peripheral nerve hyperexcitability (2 with | 0/3 (serum) |
| Morvan's syndrome) | |
| SLONM | 0/3 (serum) |
| Normal controls | 0/123 (serum) |

MG, myasthenia gravis; NMO, neuromyelitis optica; SLONM, Sporadic late onset nemaline myopathy.

Clinical Characteristics of Cavin-4 IgG Seropositive iRMD Patients

Six of eight cavin-4 IgG seropositive patients were male; median onset age was 60 years (range 18-76). Initial symptoms included rippling of muscle in lower limbs (5/8) or in all limbs, mild proximal weakness (3/8; Medical Research Council grade 4/5 in affected muscles), and isolated myalgia (1/8). Diffuse rippling ensued. Fatigue was common (7/8). Cardiac symptoms were absent. On examination, all patients had percussion-induced muscle rippling (eVideo 1) and half had percussion/stretch-induced muscle mounding. Clinical comorbidities included type 2 diabetes mellitus (1), Hashimoto thyroiditis (1), and pernicious anemia coexisting with lung biopsy-proven sarcoidosis (1).

Plasma creatine kinase was elevated in all but one patient (median 512 U/L, range: 132-2625 U/L, normal: 39-308 U/L (males), 26-192 U/L (females)). AChR binding antibodies were detected in 4/8 cases tested at diagnostic evaluation, and repetitive nerve stimulation on electrodiagnostic testing revealed a decrement of compound muscle action potential amplitude consistent with MG in 2 of those cases; single-fiber EMG revealed significantly abnormal jitter in a third case. Striational muscle autoantibodies were detected in 4/8 cases (50%; median titer: 7,680, range: 480-61,440), concomitant with MG in P7 only. Cancer screening, performed in 6/8 cases (75%), included computed tomography (CT) of chest/abdomen/pelvis (3), CT of chest (3), (18) F-fluorodeoxyglucose-positron emission tomography/CT (FDG-PET/CT)) (1). Breast carcinoma was detected in patient P6 6 months after iRMD symptom onset. Thymoma was not detected in any patient. Cardiac evaluation was performed in 3 patients: 12-lead electrocardiogram (ECG) was normal in P1, demonstrated left bundle branch block in P6, with normal echocardiogram, and demonstrated 1 st degree atrio-ventricular block in P9, with normal echocardiogram.

Muscle Histopathological and Immunohistochemical Features of Cavin-4 IgG Seropositive iRMD Patients

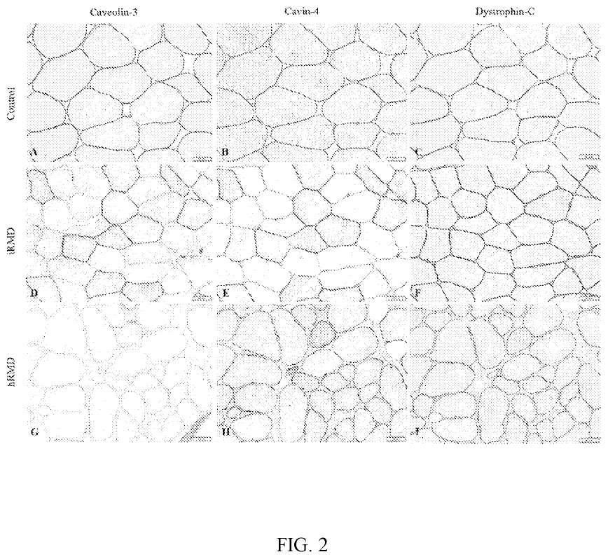

Diagnostic muscle biopsy was performed in 7 of the 8 cavin-4 IgG seropositive patients (P1-P6 and P8). Immunohistochemical studies revealed in 6 of the cases tested (P1-6) a mosaic pattern of sarcolemmal caveolin-3 and cavin-4 immunoreactivity in contrast to the uniform sarcolemmal immunoreactivity for dystrophin-C terminal peptide (FIG. 2, panels D-F). In sequential sections, fibers with absent or attenuated caveolin-3 immunoreactivity matched those with attenuated cavin-4 immunoreactivity. The percentage of muscle fibers (per low power field) with deficient sarcolemmal caveolin-3 and cavin-4 immunoreactivity ranged from 55% to 91% (median 79%). A reduction in cavin-4 expression in seropositive patient muscle was demonstrated additionally in muscle lysates by western blot (FIG. 5). Compared to iRMD muscle biopsies, hRMD muscle biopsies showed a diffuse attenuation (severely reduction or absence) of sarcolemmal caveolin-3 immunoreactivity but normal cavin-4 and dystrophin immunoreactivities (FIG. 2, panels G-I). Immunohistochemical studies performed on healthy control muscle biopsies demonstrated normal sarcolemmal dystrophin, caveolin-3, and cavin-4 immunoreactivities (FIG. 2, panels A-C).

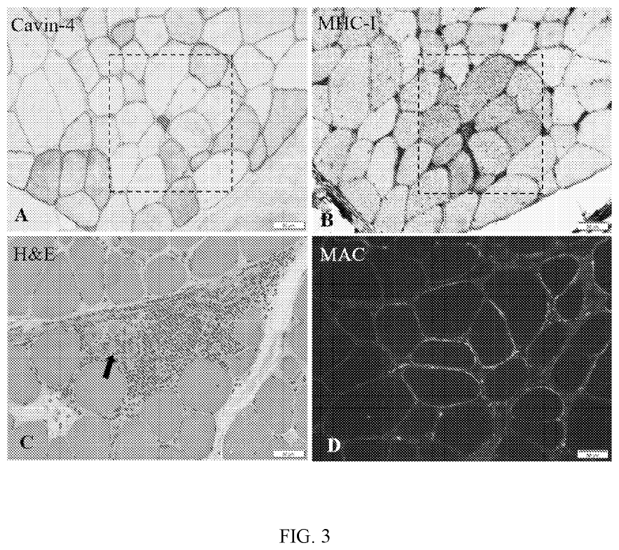

Only 29% of iRMD cases (2/7) had a muscle biopsy showing inflammation (FIG. 3), which was described as rare (minimal scattered inflammation) or mild (1 small collection per 5×-power field). Inflammation was localized to endomysial and perimysial perivascular regions (1) or perimysial perivascular regions (1). No autoaggressive inflammatory reaction was detected. Four biopsies had active myopathic features (muscle fiber necrosis and regeneration) which was minimal (3) or moderate (1) in severity.

A variable number of fibers in all iRMD muscle biopsies exhibited upregulation of sarcolemmal MHC-I and MAC deposition on non-necrotic fibers (FIG. 3B, D). Congo red stain did not detect amyloid deposition in any muscle biopsy, including those of the 2 patients with monoclonal gammopathy.

Treatment and Outcomes of Cavin-4 IgG Seropositive iRMD Patients

Immunotherapy with one or more agents was utilized in the illness course in 6/8 cases (75%), the regimens being individualized by provider preference: intravenous immune globulin (IVIG, n=4), oral prednisone (n=4) or intravenous methylprednisolone (IVMP, n=1), plasmapheresis (n=1) and azathioprine (n=2, both with MG). The previously reported patient with breast cancer (P6) was treated with lumpectomy, regional radiation, and tamoxifen in addition to IVIG 2 g/kg monthly for 2 cycles and prednisone 50 mg tapering to 5 mg over 6 months, with complete remission of muscle rippling and weakness. At last follow-up, two additional patients similarly achieved complete remission (P1 within 4 months of treatment with prednisone 20 mg daily, IVIG 2 g/kg monthly and azathioprine 2.5 mg/kg daily; P8 within 6 months of treatment with prednisone 20 mg daily and azathioprine 2.5 mg/kg daily). Rippling was mildly better in P3 (within 6 months of 1 course of plasmapheresis, followed by 2 months of IVIG 0.4 g/kg weekly, then 3 months of IVMP 500 mg weekly), and two were immunotherapy-refractory but clinically stable (P7 had co-existing MG had which significantly improved with prednisone up to 50 mg daily followed by gradual taper while rippling stabilized; P2 received only IVIG 0.4 g/kg biweekly for 2 years without significant improvement, declining other immunotherapies thereafter).

Cavin-4 IgG Seronegative iRMD Patients

Neither of the two cavin-4 IgG seronegative patients (P9 and P10) had a variant in the CAV3 or CAVIN1 genes. They had both presented with myalgia and fatigue; P10 had proximal weakness. Diffuse muscle rippling (sparing bulbar muscles) developed in both patients, and both had percussion-induced muscle rippling and muscle mounding. Neither had electrophysiological evidence of a neuromuscular transmission defect or radiological evidence of cancer.

Only P9 had a mosaic pattern of cavin-4 and caveolin-3 immunostaining with a low percentage (18%) of muscle fibers showing attenuation of cavin-4 sarcolemmal immunoreactivity, and in this patient cavin-4-IgG was detected by retrospective PhIP Seq, but not by other validation studies. Neither muscle biopsy contained evidence of an inflammatory exudate, but one had rare necrotic and regenerating muscle fibers, and both had patchy upregulation of sarcolemmal MHC-1 and MAC deposition. In support of his disease having an immune-mediated basis, P9 experienced complete symptom remission within 1 month after IVMP 500 mg weekly for 4 weeks, with maintenance on IVIG 0.4 g/kg and azathioprine 2 mg/kg daily thereafter. In the year prior to evaluation at our institution, P10 received IVIG (2 g/kg over 5 days, then monthly 1 g/kg for two courses), without improvement; she has since remained clinically stable on monthly IVIG (2 g/kg) over short-term follow-up.

Together, these results demonstrate that a cavin-4 polypeptide is the antigenic target of iRMD-specific autoantibodies, and that a cavin-4 polypeptide fragment (e.g., a recombinant cavin-4 polypeptide fragment) can be used to detect the presence of iRMD-specific autoantibodies (e.g., anti-cavin-4 polypeptide antibodies) in samples (e.g., serum samples).

### Example 2: Identifying iRMD

A blood sample (e.g., serum) is obtained from a human having RMD. The obtained sample is contacted with a fragment of a cavin-4 polypeptide to form an antibody-cavin-4 fragment complex if the sample contains anti-cavin-4 polypeptide autoantibodies (e.g., iRMD-specific autoantibodies), and then examined for the presence of an antibody-cavin-4 fragment complex.

If antibody-cavin-4 fragment complexes are detected in the sample, then the human is classified as having an iRMD.

If antibody-cavin-4 fragment complexes are not detected in the sample, then the human is classified as having a RMD that is not immune-mediated.

### Example 3: Treating iRMD

A sample (e.g., serum) is obtained from a human having RMD. The sample is assessed for the presence or absence of anti-cavin-4 polypeptide autoantibodies (e.g., iRMD-specific autoantibodies) by contacting the sample with a fragment of a cavin-4 polypeptide to form an antibody-cavin-4 fragment complex if the sample contains anti-cavin-4 polypeptide autoantibodies (e.g., iRMD-specific autoantibodies), and then examined for the presence of an antibody-cavin-4 fragment complex.

When the presence of anti-cavin-4 polypeptide autoantibodies (e.g., iRMD-specific autoantibodies) is detected in the sample, then the human is identified as having an iRMD, and is administered one or more immunosuppressants (e.g., rituximab, mycophenolate, and azathioprine), is administered one or more steroids (e.g., corticosteroids such as oral prednisone and IV methylprednisolone), is subjected to IVIG, and/or is subjected to plasma exchange therapy.

### Example 4: Treating RMD

A sample (e.g., serum) is obtained from a human having RMD. The sample is assessed for the presence or absence of anti-cavin-4 polypeptide autoantibodies (e.g., iRMD-specific autoantibodies) by contacting the sample with a fragment of a cavin-4 polypeptide to form an antibody-cavin-4 fragment complex if the sample contains anti-cavin-4 polypeptide autoantibodies (e.g., iRMD-specific autoantibodies), and then examined for the presence of an antibody-cavin-4 fragment complex.

When the absence of anti-cavin-4 polypeptide autoantibodies (e.g., iRMD-specific autoantibodies) is detected in the sample, then the human is identified as having RMD that is not immune-mediated, and is administered one or more RMD treatments that are not immunosuppressants (e.g., weight control therapy to avoid obesity and physical therapy to promote mobility and prevent contractures).

### Example 5: Exemplary Embodiments

Embodiment 1. A method for determining whether or not a mammal has an immune-mediated caveolinopathy disease, wherein said method comprises:
(a) contacting a serum sample from said mammal with a composition comprising an antigen to form an antigen-anti-caveolae-associated protein 4 (cavin-4) polypeptide autoantibody complex if said serum sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(b) detecting the presence or absence of said complex, wherein the presence of said complex indicates that said mammal has said immune-mediated caveolinopathy disease, and wherein the absence of said complex indicates that said mammal does not have said immune-mediated caveolinopathy disease.

Embodiment 2. The method of claim 1, wherein said composition comprises a cell lysate obtained from a cell (a) comprising exogenous nucleic acid encoding said antigen and (b) expressing said antigen.

Embodiment 3. The method of any one of claims 1-2, wherein said antigen is said cavin-4 polypeptide.

Embodiment 4. The method of any one of claims 1-2, wherein said antigen is said variant.

Embodiment 5. The method of any one of claims 1-2, wherein said antigen is said fragment.

Embodiment 6. The method of claim 5, wherein said fragment consists of the amino acid sequence set forth in any one of SEQ ID NOs: 1-3.

Embodiment 7. The method of any one of claims 1-6, wherein said antigen is covalently linked to a detectable label.

Embodiment 8. The method of claim 7, wherein said detectable label is selected from the group consisting of a green fluorescent protein (GFP) polypeptide, tetramethylrhodamine isothiocyanate (TRITC), fluorescein isothiocyanate (FITC), a poly(His) tag, a glutathione-S-transferase (GST) tag, and biotin.

Embodiment 9. The method of any one of claims 1-8, wherein said detecting comprises an immunological assay.

Embodiment 10. The method of any one of claims 1-9, wherein said mammal is a human.

Embodiment 11. The method of any one of claims 1-10, wherein said method comprises detecting the presence of said complex.

Embodiment 12. The method of claim 11, wherein said method comprises classifying said mammal as having said immune-mediated caveolinopathy disease.

Embodiment 13. The method of any one of claims 1-10, wherein said method comprises detecting the absence of said complex.

Embodiment 14. The method of claim 13, wherein said method comprises classifying said mammal as not having said immune-mediated caveolinopathy disease.

Embodiment 15. The method of any one of claims 1-14, wherein said immune-mediated caveolinopathy disease is immune-mediated rippling muscle disease (iRMD).

Embodiment 16. A method for determining whether or not a mammal has an immune-mediated caveolinopathy disease, wherein said method comprises:
(a) detecting the presence or absence a decreased level of a cavin-4 polypeptide in a muscle tissue sample from said mammal; and
(b) classifying said mammal as having said immune-mediated caveolinopathy disease if said presence is determined; and
(c) classifying said mammal as not having said immune-mediated caveolinopathy disease if said absence is determined.

Embodiment 17. The method of claim 16, wherein said detecting comprises an immunological assay.

Embodiment 18. The method of any one of claims 16-17, wherein said mammal is a human.

Embodiment 19. The method of any one of claims 16-18, wherein said muscle tissue sample is a skeletal muscle sample or a cardiac muscle tissue sample.

Embodiment 20. The method of any one of claims 16-19, wherein said method comprises detecting the presence of said decreased level.

Embodiment 21. The method of claim 20, wherein said method comprises classifying said mammal as having said immune-mediated caveolinopathy disease.

Embodiment 22. The method of any one of claims 16-19, wherein said method comprises detecting the absence of said decreased level.

Embodiment 23. The method of claim 22, wherein said method comprises classifying said mammal as not having said immune-mediated caveolinopathy disease.

Embodiment 24. The method of any one of claims 16-23, wherein said immune-mediated caveolinopathy disease is iRMD.

Embodiment 25. A method for treating a mammal having an immune-mediated caveolinopathy disease, wherein said method comprises:
(a) determining that a serum sample from said mammal comprises the presence of anti-cavin-4 polypeptide autoantibodies or a muscle tissue sample from said mammal comprises the presence of a decreased level of a cavin-4 polypeptide, and
(b) administering an immunosuppressant to said mammal.

Embodiment 26. The method of claim 25, wherein said determining that said serum sample from said mammal comprises the presence of anti-cavin-4 polypeptide autoantibodies comprises:
(i) contacting said serum sample with a composition comprising an antigen to form an antigen-anti-cavin-4 polypeptide autoantibody complex if said sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(ii) detecting the presence of said complex, thereby determining that said mammal comprises the presence of said anti-cavin-4 polypeptide autoantibodies.

Embodiment 27. The method of claim 26, wherein said composition comprises a cell lysate obtained from a cell (a) comprising exogenous nucleic acid encoding said antigen and (b) expressing said antigen.

Embodiment 28. The method of any one of claims 26-27, wherein said antigen is said cavin-4 polypeptide.

Embodiment 29. The method of any one of claims 26-27, wherein said antigen is said variant.

Embodiment 30. The method of any one of claims 26-27, wherein said antigen is said fragment.

Embodiment 31. The method of claim 30, wherein said fragment consists of the amino acid sequence set forth in any one of SEQ ID NOs: 1-3.

Embodiment 32. The method of any one of claims 26-31, wherein said antigen is covalently linked to a detectable label.

Embodiment 33. The method of claim 32, wherein said detectable label is selected from the group consisting of a GFP polypeptide, TRITC, FITC, a poly(His) tag, a GST tag, and biotin.

Embodiment 34. The method of claim 25, wherein said determining that said muscle tissue sample from said mammal comprises the presence of said decreased level of said cavin-4 polypeptide comprises:
(i) contacting said muscle tissue sample with a polypeptide having the ability to bind to said cavin-4 polypeptide; and
(ii) detecting the presence of said decreased level of said cavin-4 polypeptide, thereby determining that said muscle tissue sample from said mammal comprises the presence of said decreased level of said cavin-4 polypeptide.

Embodiment 35. The method of any one of claims 25-34, wherein said mammal is a human.

Embodiment 36. The method of any one of claims 25-35, wherein said immune-mediated caveolinopathy disease is iRMD.

Embodiment 37. The method of any one of claims 25-36, wherein said immunosuppressant is selected from the group consisting of rituximab, mycophenolate, and azathioprine.

Embodiment 38. A method for treating an immune-mediated caveolinopathy disease, wherein said method comprises administering an immunosuppressant to a mammal that was identified as having a caveolinopathy disease and anti-cavin-4 polypeptide autoantibodies.

Embodiment 39. The method of claim 38, wherein said identifying comprises:
(a) contacting a sample from said mammal with a composition comprising an antigen to form an antigen-anti-cavin-4 polypeptide autoantibody complex if said sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(b) detecting the presence of said complex, thereby identifying said mammal as having said anti-cavin-4 polypeptide autoantibodies.

Embodiment 40. The method of claim 39, wherein said composition comprises a cell lysate obtained from a cell (a) comprising exogenous nucleic acid encoding said antigen and (b) expressing said antigen.

Embodiment 41. The method of any one of claims 39-40, wherein said antigen is said cavin-4 polypeptide.

Embodiment 42. The method of any one of claims 39-40, wherein said antigen is said variant.

Embodiment 43. The method of any one of claims 39-40, wherein said antigen is said fragment.

Embodiment 44. The method of claim 43, wherein said fragment consists of the amino acid sequence set forth in any one of SEQ ID NOs: 1-3.

Embodiment 45. The method of any one of claims 39-44, wherein said antigen is covalently linked to a detectable label.

Embodiment 46. The method of claim 45, wherein said detectable label is selected from the group consisting of a GFP polypeptide, TRITC, FITC, a poly(His) tag, a GST tag, and biotin.

Embodiment 47. The method of any one of claims 38-46, wherein said mammal is a human.

Embodiment 48. The method of any one of claims 38-47, wherein said sample is a serum sample.

Embodiment 49. The method of any one of claims 38-48, wherein said immune-mediated caveolinopathy disease is iRMD.

Embodiment 50. The method of any one of claims 38-49, wherein said immunosuppressant is selected from the group consisting of rituximab, mycophenolate, and azathioprine.

Embodiment 51. A method for treating a mammal having a caveolinopathy disease, wherein said method comprises:
(a) determining that a serum sample from said mammal lacks the presence of anti-cavin-4 polypeptide autoantibodies or that a muscle sample from said mammal lacks a decreased level of a cavin-4 polypeptide, and
(b) administering a therapy for said caveolinopathy disease to said mammal, wherein said therapy is not an immunosuppressant.

Embodiment 52. The method of claim 51, wherein said determining that said serum sample from said mammal lacks the presence of anti-cavin-4 polypeptide autoantibodies comprises:
(i) contacting said serum sample with a composition comprising an antigen to form an antigen-anti-cavin-4 polypeptide autoantibody complex if said serum sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(ii) detecting the absence of said complex, thereby that said serum sample from said mammal lacks the presence of anti-cavin-4 polypeptide autoantibodies.

Embodiment 53. The method of claim 52, wherein said composition comprises a cell lysate obtained from a cell (a) comprising exogenous nucleic acid encoding said antigen and (b) expressing said antigen.

Embodiment 54. The method of any one of claims 52-53, wherein said antigen is said cavin-4 polypeptide.

Embodiment 55. The method of any one of claims 52-53, wherein said antigen is said variant.

Embodiment 56. The method of any one of claims 52-53, wherein said antigen is said fragment.

Embodiment 57. The method of claim 56, wherein said fragment consists of the amino acid sequence set forth in any one of SEQ ID NOs: 1-3.

Embodiment 58. The method of any one of claims 52-57, wherein said antigen is covalently linked to a detectable label.

Embodiment 59. The method of claim 58, wherein said detectable label is selected from the group consisting of a GFP polypeptide, TRITC, FITC, a poly(His) tag, a GST tag, and biotin.

Embodiment 60. The method of claim 51, wherein said determining that said muscle tissue sample from said mammal lacks the presence of said decreased level of said cavin-4 polypeptide comprises:
(i) contacting said muscle tissue sample with a polypeptide having the ability to bind to said cavin-4 polypeptide; and
(ii) detecting an absence of said decreased level of said cavin-4 polypeptide, thereby determining that said muscle tissue sample from said mammal lacks the presence of said decreased level of said cavin-4 polypeptide.

Embodiment 61. The method of any one of claims 51-60, wherein said mammal is a human.

Embodiment 62. The method of any one of claims 51-61, wherein said caveolinopathy disease is RMD that is not immune-mediated.

Embodiment 63. The method of any one of claims 51-62, wherein said therapy for said caveolinopathy disease is selected from the group consisting of a weight control therapy and physical therapy.

Embodiment 64. A method for treating a caveolinopathy disease, wherein said method comprises administering a therapy for said caveolinopathy disease to a mammal that was identified as lacking anti-cavin-4 polypeptide autoantibodies, wherein said therapy is not an immunosuppressant.

Embodiment 65. The method of claim 64, wherein said identifying comprises:
(a) contacting a sample from said mammal with a composition comprising an antigen to form an antigen-anti-cavin-4 polypeptide autoantibody complex if said sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(b) detecting the absence of said complex, thereby identifying said mammal as lacking anti-cavin-4 polypeptide autoantibodies.

Embodiment 66. The method of claim 65, wherein said composition comprises a cell lysate obtained from a cell (a) comprising exogenous nucleic acid encoding said antigen and (b) expressing said antigen.

Embodiment 67. The method of any one of claims 65-66, wherein said antigen is said cavin-4 polypeptide.

Embodiment 68. The method of any one of claims 65-66, wherein said antigen is said variant.

Embodiment 69. The method of any one of claims 65-66, wherein said antigen is said fragment.

Embodiment 70. The method of claim 69, wherein said fragment consists of the amino acid sequence set forth in any one of SEQ ID NOs: 1-3.

Embodiment 71. The method of any one of claims 65-70, wherein said antigen is covalently linked to a detectable label.

Embodiment 72. The method of claim 71, wherein said detectable label is selected from the group consisting of a GFP polypeptide, TRITC, FITC, a poly(His) tag, a GST tag, and biotin.

Embodiment 73. The method of any one of claims 64-72, wherein said mammal is a human.

Embodiment 74. The method of any one of claims 64-73, wherein said sample is a serum sample.

Embodiment 75. The method of any one of claims 64-74, wherein said caveolinopathy disease is RMD that is not immune-mediated.

Embodiment 76 The method of any one of claims 64-75, wherein said therapy for said caveolinopathy disease is selected from the group consisting of a weight control therapy and physical therapy.

Embodiment 77. A method for treating an immune-mediated caveolinopathy disease, wherein said method comprises:
(a) removing blood from a mammal having said immune-mediated caveolinopathy disease, thereby obtaining removed blood,
(b) removing at least some anti-cavin-4 polypeptide autoantibodies from said removed blood, thereby obtaining processed blood, and
(c) reintroducing said processed blood into said mammal.

Embodiment 78. The method of claim 77, wherein said step (b) comprises contacting said removed blood with immobilized antigen, wherein said antigen is a cavin-4 polypeptide, a fragment of said cavin-4 polypeptide having the ability to bind to an anti-cavin-4 polypeptide autoantibody, or a variant of said cavin-4 polypeptide having the ability to bind to an anti-cavin-4 polypeptide autoantibody.

Embodiment 79. The method of claim 78, wherein said antigen is said cavin-4 polypeptide.

Embodiment 80. The method of claim 78, wherein said antigen is said variant.

Embodiment 81. The method of claim 78, wherein said antigen is said fragment.

Embodiment 82. The method of claim 81, wherein said fragment consists of the amino acid sequence set forth in any one of SEQ ID NOs: 1-3.

Embodiment 83. The method of any one of claims 78-82, wherein said mammal is a human.

Embodiment 84. A method for treating a mammal having an immune-mediated caveolinopathy disease, wherein said method comprises:
administering to said mammal, a polypeptide comprising (i) a cavin-4 polypeptide, a fragment of said cavin-4 polypeptide having the ability to bind to an anti-cavin-4 polypeptide autoantibody, or a variant of said cavin-4 polypeptide having the ability to bind to an anti-cavin-4 polypeptide autoantibody, and (ii) an IgG1-derived Fc fragment.

Embodiment 85. The method of claim 84, wherein said IgG1-derived Fc fragment comprises a dimeric IgG1-derived Fc fragment.

Embodiment 86. The method of any one of claims 84-85, wherein said IgG1-derived Fc fragment is a human IgG1-derived Fc fragment.

Embodiment 87. The method of any one of claims 84-86, wherein said polypeptide comprises said cavin-4 polypeptide.

Embodiment 88. The method of any one of claims 84-86, wherein said polypeptide comprises said variant.

Embodiment 89. The method of any one of claims 84-86, wherein said polypeptide comprises said fragment.

Embodiment 90. The method of claim 89, wherein said fragment consists of the amino acid sequence set forth in any one of SEQ ID NOs: 1-3.

Embodiment 91. The use of a composition comprising an antigen to treat an immune-mediated caveolinopathy disease, wherein said antigen is a cavin-4 polypeptide, a fragment of said cavin-4 polypeptide having the ability to bind to an anti-cavin-4 polypeptide autoantibody, or a variant of said cavin-4 polypeptide having the ability to bind to an anti-cavin-4 polypeptide autoantibody.

Embodiment 92. The use of a composition comprising an antigen to treat an immune-mediated caveolinopathy disease, wherein said antigen is a cavin-4 polypeptide, a fragment of said cavin-4 polypeptide having the ability to bind to an anti-cavin-4 polypeptide autoantibody, or a variant of said cavin-4 polypeptide having the ability to bind to an anti-cavin-4 polypeptide autoantibody, wherein said use comprises removing at least some anti-cavin-4 polypeptide autoantibodies from the blood of a mammal having said immune-mediated caveolinopathy disease.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-21. (canceled)

22. A method for treating a mammal having an immune-mediated caveolinopathy disease, wherein said method comprises:
(a) determining that a serum sample from said mammal comprises the presence of anti-cavin-4 polypeptide autoantibodies or a muscle tissue sample from said mammal comprises the presence of a decreased level of a cavin-4 polypeptide, and
(b) administering an immunosuppressant to said mammal.

23. The method of claim 22, wherein said determining that said serum sample from said mammal comprises the presence of anti-cavin-4 polypeptide autoantibodies comprises:
(i) contacting said serum sample with a composition comprising an antigen to form an antigen-anti-cavin-4 polypeptide autoantibody complex if said sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(ii) detecting the presence of said complex, thereby determining that said mammal comprises the presence of said anti-cavin-4 polypeptide autoantibodies.

24. The method of claim 23, wherein said composition comprises a cell lysate obtained from a cell (a) comprising exogenous nucleic acid encoding said antigen and (b) expressing said antigen.

25. The method of claim 22, wherein said determining that said muscle tissue sample from said mammal comprises the presence of said decreased level of said cavin-4 polypeptide comprises:
(i) contacting said muscle tissue sample with a polypeptide having the ability to bind to said cavin-4 polypeptide; and
(ii) detecting the presence of said decreased level of said cavin-4 polypeptide, thereby determining that said muscle tissue sample from said mammal comprises the presence of said decreased level of said cavin-4 polypeptide.

26. A method for treating an immune-mediated caveolinopathy disease,
wherein said method comprises administering an immunosuppressant to a mammal that was identified as having a caveolinopathy disease and anti-cavin-4 polypeptide autoantibodies.

27. The method of claim 26, wherein said identifying comprises:
(a) contacting a serum sample from said mammal with a composition comprising an antigen to form an antigen-anti-cavin-4 polypeptide autoantibody complex if said sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(b) detecting the presence of said complex, thereby identifying said mammal as having said anti-cavin-4 polypeptide autoantibodies.

28. The method of claim 27, wherein said composition comprises a cell lysate obtained from a cell (a) comprising exogenous nucleic acid encoding said antigen and (b) expressing said antigen.

29. The method of claim 26, wherein said fragment consists of the amino acid sequence set forth in any one of SEQ ID NOs: 1-3.

30. The method of claim 26, wherein said antigen is covalently linked to a detectable label.

31. The method of claim 30, wherein said detectable label is selected from the group consisting of a GFP polypeptide, TRITC, FITC, a poly(His) tag, a GST tag, and biotin.

32. The method of claim 26, wherein said mammal is a human.

33. The method of claim 26, wherein said immune-mediated caveolin Pathy disease is iRMD.

34. The method of claim 26, wherein said immunosuppressant is selected from the group consisting of rituximab, mycophenolate, and azathioprine.

35. A method for treating a mammal having a caveolinopathy disease, wherein said method comprises:
(a) determining that a serum sample from said mammal lacks the presence of anti-cavin-4 polypeptide autoantibodies or that a muscle sample from said mammal lacks a decreased level of a cavin-4 polypeptide, and
(b) administering a therapy for said caveolinopathy disease to said mammal, wherein said therapy is not an immunosuppressant.

36. The method of claim 35, wherein said determining that said serum sample from said mammal lacks the presence of anti-cavin-4 polypeptide autoantibodies comprises:
(i) contacting said serum sample with a composition comprising an antigen to form an antigen-anti-cavin-4 polypeptide autoantibody complex if said serum sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(ii) detecting the absence of said complex, thereby that said serum sample from said mammal lacks the presence of anti-cavin-4 polypeptide autoantibodies.

37. The method of claim 36, wherein said composition comprises a cell lysate obtained from a cell (a) comprising exogenous nucleic acid encoding said antigen and (b) expressing said antigen.

38. The method of claim 35, wherein said determining that said muscle tissue sample from said mammal lacks the presence of said decreased level of said cavin-4 polypeptide comprises:
(i) contacting said muscle tissue sample with a polypeptide having the ability to bind to said cavin-4 polypeptide; and
(ii) detecting an absence of said decreased level of said cavin-4 polypeptide, thereby determining that said muscle tissue sample from said mammal lacks the presence of said decreased level of said cavin-4 polypeptide.

39. A method for treating a caveolinopathy disease, wherein said method comprises administering a therapy for said caveolinopathy disease to a mammal that was identified as lacking anti-cavin-4 polypeptide autoantibodies, wherein said therapy is not an immunosuppressant.

40. The method of claim 39, wherein said identifying comprises:
(a) contacting a serum sample from said mammal with a composition comprising an antigen to form an antigen-anti-cavin-4 polypeptide autoantibody complex if said sample comprises an anti-cavin-4 polypeptide autoantibody, wherein said antigen is a cavin-4 polypeptide, a fragment of said cavin-4 polypeptide having the ability to bind to said anti-cavin-4 polypeptide autoantibody, or a variant of said cavin-4 polypeptide having the ability to bind to said anti-cavin-4 polypeptide autoantibody; and
(b) detecting the absence of said complex, thereby identifying said mammal as lacking anti-cavin-4 polypeptide autoantibodies.

41. The method of claim 40, wherein said composition comprises a cell lysate obtained from a cell (a) comprising exogenous nucleic acid encoding said antigen and (b) expressing said antigen.

42. The method of claim 39, wherein said fragment consists of the amino acid sequence set forth in any one of SEQ ID NOs: 1-3.

43. The method of claim 39, wherein said antigen is covalently linked to a detectable label.

44. The method of claim 43, wherein said detectable label is selected from the group consisting of a GFP polypeptide, TRITC, FITC, a poly(His) tag, a GST tag, and biotin.

45. The method of claim 39, wherein said mammal is a human.

46. The method of claim 39, wherein said caveolinopathy disease is RMD that is not immune-mediated.

47. The method of claim 39, wherein said therapy for said caveolinopathy disease is selected from the group consisting of a weight control therapy and physical therapy.

48. A method for treating an immune-mediated caveolinopathy disease, wherein said method comprises:
(a) removing blood from a mammal having said immune-mediated caveolinopathy disease, thereby obtaining removed blood,
(b) removing at least some anti-cavin-4 polypeptide autoantibodies from said removed blood, thereby obtaining processed blood, and
(c) reintroducing said processed blood into said mammal.

49. The method of claim 48, wherein said step (b) comprises contacting said removed blood with immobilized antigen, wherein said antigen is a cavin-4 polypeptide, a fragment of said cavin-4 polypeptide having the ability to bind to an anti-cavin-4 polypeptide autoantibody, or a variant of said cavin-4 polypeptide having the ability to bind to an anti-cavin-4 polypeptide autoantibody.

50. The method of claim 49, wherein said fragment consists of the amino acid sequence set forth in any one of SEQ ID NOs: 1-3.

51. The method of claim 48, wherein said mammal is a human.

52. A method for treating a mammal having an immune-mediated caveolinopathy disease, wherein said method comprises:
administering to said mammal, a polypeptide comprising (i) a cavin-4 polypeptide, a fragment of said cavin-4 polypeptide having the ability to bind to an anti-cavin-4 polypeptide autoantibody, or a variant of said cavin-4 polypeptide having the ability to bind to an anti-cavin-4 polypeptide autoantibody, and (ii) an IgG1-derived Fc fragment.

53. The method of claim 52, wherein said IgG1-derived Fc fragment comprises a dimeric IgG1-derived Fc fragment.

54. The method of claim 52, wherein said IgG1-derived Fc fragment is a human IgG1-derived Fc fragment.

55. The method of claim 52, wherein said fragment consists of the amino acid sequence set forth in any one of SEQ ID NOs: 1-3.

56-57. (canceled)


SEQUENCE LISTING (4 sequences)

SEQ ID NO: 1 - protein, 50 aa, synthetic construct
SLSVVKDRNLTENQEEDDDDIFDPPVDLSSDEEYYVEESRSARLRKSGKE

SEQ ID NO: 2 - protein, 51 aa, synthetic construct
PSKEAFKMRSLRKGKDRTVAEGEECAREMGVDIIARSESLGPISELYSDEL

SEQ ID NO: 3 - protein, 50 aa, synthetic construct
CAREMGVDIIARSESLGPISELYSDELSEPEHEAARPVYPPHEGREIPTP

SEQ ID NO: 4 - protein, 364 aa, Homo sapiens
MEHNGSASNADKIHQNRLSSVTEDEDQDAALTIVTVLDKVASIVDSVQASQKRIEERHREMENAIKSVQIDLLKLSQSHSNTGHIINKLFEKTRKVSAHIKDVKARVEKQQIHVKKVEVKQEEIMKKNKFRVVIFQEKFRCPTSLSVVKDRNLTENQEEDDDDIFDPPVDLSSDEEYYVEESRSARLRKSGKEHIDNIKKAFSKENMQKTRQNLDKKVNRIRTRIVTPERRERLRQSGERLRQSGERLRQSGERFKKSISNAAPSKEAFKMRSLRKGKDRTVAEGEECAREMGVDIIARSESLGPISELYSDELSEPEHEAARPVYPPHEGREIPTPEPLKVTFKSQVKVEDDESLLLDLKHSS